(19) United States
(12) Patent Application Publication
(10) Pub. No.: US 2017/0196888 A1
(43) Pub. Date: Jul. 13, 2017

(54) COMPOSITIONS AND METHODS FOR TREATING CANCER

(71) Applicant: Yeda Research and Development Co. Ltd., Rehovot (IL)

(72) Inventors: Yosef YARDEN, Rehovot (IL); Mattia LAURIOLA, Rehovot (IL)

(21) Appl. No.: 15/321,281

(22) PCT Filed: Jun. 25, 2015
(86) PCT No.: PCT/IL15/50661

§ 371 Date: Dec. 22, 2016

Related U.S. Application Data
(60) Provisional application No. 62/016,745, filed on Jun. 25, 2014.

(51) Int. Cl. A61K 31/573; A61K 9/00; A61K 31/517

(52) U.S. Cl. CPC A61K 31/573; A61K 31/517; A61K 9/0053

(57) ABSTRACT

A method of treating cancer in a subject in need thereof is provided. The method comprising administering to the subject a receptor tyrosine kinase (RTK)-specific cancer therapy and a glucocorticoid or a glucocorticoid analog, such that an efficacy window of said RTK-specific cancer therapy and an efficacy window of said glucocorticoid or glucocorticoid analog substantially overlap.

### FIELD AND BACKGROUND OF THE INVENTION

The present invention, in some embodiments thereof, relates to compositions and methods for treating cancer.

Growth factors acting through receptor tyrosine kinases (RTKs), along with steroid hormones acting through nuclear receptors (NRs), critically regulate cell-to-cell interactions in development and throughout adulthood. For example, type I RTKs (also called ERBB or HER) and their ligands of the epidermal growth factor (EGF) family regulate ductal and alveolar morphogenesis of the mammary gland¹. Similarly, the NR called glucocorticoid receptor (GR) controls cell proliferation during lobulo-alveolar development of the mammary gland². Despite recruitment of very different routes of signal transduction, RTKs and NRs maintain extensive crosstalk, but the physiological integration and effects of this crosstalk on body homeostasis remain incompletely understood.

One prototype RTK is the EGF-receptor (EGFR/ERBB1). In addition to EGF, EGFR binds several growth factors, including transforming growth factor alpha (TGFα) and the heparin-binding EGF-like growth factor (HB-EGF)³. Integration of EGF-induced signals culminates in a wave-like pattern of transcription⁴: in response to EGF, a group of microRNAs undergoes rapid downregulation, and concurrently their target transcripts, which encode immediate early transcription factors (IETFs), and other immediate early genes (IEGs), are activated. Subsequent transcription of delayed early genes (DEGs), a group encoding transcriptional repressors and negative feedback regulators, such as MAPK phosphatases (DUSPs) and ERRFI1/MIG6, which promotes degradation and inhibits self-phosphorylation of EGFR⁵, regulates expression of late, fate-determining genes.

In analogy to RTKs, the biological actions of glucocorticoids (GCs), as well as other steroid hormones, are mediated by ubiquitously expressed receptors of the NR superfamily⁶. GCs are synthesized in the adrenal gland and are delivered through systemic circulation to GRs⁷. Once in the nucleus, ligand-bound GRs activate transcription by binding to specific DNA elements, called glucocorticoid response elements (GREs). Alternatively, GR mediates direct repression of specific genes by binding to negative GREs (nGREs)⁸ or by altering chromatin status⁹. Yet an additional mechanism of regulation involves tethered transrepression by physical complex formation between GRs and other TFs, such as STATS¹⁰. These modes of regulation mediate both pro-survival effects on epithelial cells, and induction of apoptosis of lymphoid and myeloid cells, which led to the approval of a GC analog, some 50 years ago, for treatment of childhood leukemia¹¹.

Interestingly, GCs were found to mediate a negative growth effect on EGF responsive cells via Gene 33, a natural negative inhibitor of EGFR signalling. It was therefore suggested that Gene 33 may function in the cross-talk between EGF signalling and other mitogenic and/or stress signalling pathways (Xu et al. J Biol Chem. 2005 Jan. 28; 280(4):2924-33). GCs are also widely used as co-medication of various carcinomas, due to their ability to reduce toxicity of chemotherapy.

Interestingly, in vitro studies combining GC treatment with monoclonal antibody therapy to HER2 on breast cancer cell lines have found inhibition of the anti-tumor activity of the anti HER2 antibody. The study concluded that chemotherapeutic regimens should be effected without glucocorticoid premedication (Sumikawa et al. Int. J. Oncol. 2008 March;32(3):683-8).

Wagenblast J et al. reported similar findings in head and neck cancer cell lines treated with Cetuximab and dexamethasone Oncol Rep. 2009 July; 22(1):171-6.

Hence, the common paradigm, to date, is that glucocorticoid treatment suppresses the growth inhibitory effects of RTK-specific therapy.

### SUMMARY OF THE INVENTION

According to an aspect of some embodiments of the present invention there is provided a method of treating cancer in a subject in need thereof, the method comprising administering to the subject a receptor tyrosine kinase (RTK)-specific cancer therapy and a glucocorticoid or a glucocorticoid analog, such that an efficacy window of the RTK-specific cancer therapy and an efficacy window of the glucocorticoid or glucocorticoid analog substantially overlap.

According to an aspect of some embodiments of the present invention there is provided a composition-of-matter comprising a therapeutically effective amount of an RTK-specific cancer therapy and a therapeutically effective amount of a glucocorticoid or glucocorticoid analog, the composition being such that an efficacy window of the RTK-specific cancer therapy and an efficacy window of the glucocorticoid or glucocorticoid analog substantially overlap.

According to an aspect of some embodiments of the present invention there is provided an article of manufacture identified for the treatment of cancer comprising, in separate containers, a therapeutically effective amount of an RTK-specific cancer therapy and a therapeutically effective amount of a glucocorticoid or glucocorticoid analog.

According to some embodiments of the invention, each of the therapeutically effective amount of RTK-specific cancer therapy and the therapeutically effective amount of the glucocorticoid or glucocorticoid analog is effective in treating cancer.

According to some embodiments of the invention, the RTK-specific cancer therapy is conjugated to the glucocorticoid or glucocorticoid analog.

According to some embodiments of the invention, the RTK-specific cancer therapy is administered paraenterally.

According to some embodiments of the invention, the glucocorticoid or analog is administered orally.

According to some embodiments of the invention, the administering is under a circadian regimen.

According to some embodiments of the invention, the regimen comprises administering the RTK-specific cancer therapy under glucocorticoid signalling activation.

According to some embodiments of the invention, the glucocorticoid signalling activation is an endogenously activated glucocorticoid signalling.

According to some embodiments of the invention, the glucocorticoid analog is selected from the group consisting of prednisone, prednisolone, fludrocortisone, and dexamethasone.

According to some embodiments of the invention, the glucocorticoid analog comprises a non-steroidal glucocorticoid receptor agonist.

According to some embodiments of the invention, the non-steroidal glucocorticoid receptor agonist is selected from the group consisting of CpdA, LGD5552, AL-438, ZK245186, ZK216348, Quinol-4-ones and BI115.

According to some embodiments of the invention, the RTK-specific cancer therapy comprises a small molecule inhibitor.

According to some embodiments of the invention, the RTK-specific cancer therapy comprises an antibody.

According to some embodiments of the invention, the RTK is selected from the group consisting of c-met, VEGFR, INSR, PDGFR, EphR, FGFR and AXL.

According to some embodiments of the invention, the RTK is an ErbB polypeptide.

According to some embodiments of the invention, the ErbB polypeptide is an EGFR.

According to some embodiments of the invention, the RTK-specific cancer therapy is selected from the group consisting of Erlotinib, Genfitinib and Lapatinib.

According to some embodiments of the invention, the RTK-specific cancer therapy is selected from the group consisting of Panitumumab and Cetuximab.

According to some embodiments of the invention, a maximal efficacy window of the RTK-specific cancer therapy and a maximal efficacy window of the glucocorticoid or glucocorticoid analog overlap for at least 10 hours.

According to some embodiments of the invention, the RTK-specific cancer therapy and the glucocorticoid or glucocorticoid analog are administered substantially simultaneously.

According to some embodiments of the invention, a plasma peak concentration of the RTK-specific cancer therapy and a plasma peak concentration of the glucocorticoid or glucocorticoid analog occur substantially simultaneously.

According to some embodiments of the invention, the RTK-specific cancer therapy and the glucocorticoid or glucocorticoid analog are administered within 12 hours of each other.

According to some embodiments of the invention, the RTK-specific cancer therapy and the glucocorticoid or glucocorticoid analog are administered within 1 hour of each other.

According to some embodiments of the invention, the cancer is not a lymphoma, prostate cancer or breast cancer.

According to some embodiments of the invention, cells of the cancer express the RTK.

According to some embodiments of the invention, cells of the cancer display activation of the RTK.

According to some embodiments of the invention, the administering results in an improvement in survival relative to a subject treated with the RTK-specific cancer therapy only.

According to some embodiments of the invention, the administering results in an improvement in progression free survival relative to a subject treated with the RTK-specific cancer therapy only.

According to some embodiments of the invention, the administering results in an improvement in overall survival relative to a subject treated with the RTK-specific cancer therapy only.

According to some embodiments of the invention, the RTK-specific cancer therapy and the glucocorticoid or glucocorticoid analog are in a single formulation.

According to some embodiments of the invention, the RTK-specific cancer therapy is conjugated to the glucocorticoid or glucocorticoid analog.

According to some embodiments of the invention, the RTK-specific cancer therapy and the glucocorticoid or glucocorticoid analog are in separate formulations.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-M show that ligand-bound GRs inhibit EGF-induced migration of mammary cells. FIG. 1a-MCF10A cells growing in transwells were treated for 16 hours with EGF (10 ng/ml), DEX (100 nM), RU486 (5 μM), or their combinations. Shown are representative crystal violet staining images of migrated cells from three experiments. FIG. 1B—Cell-covered areas from 4 microscope fields of A were determined. ***p<0.0001 (1-way Anova). FIG. 1C—Cells pre-treated with the indicated siRNA oligonucleotides were seeded in transwells, stimulated as shown and 16 hours later migrated cells were photographed. FIG. 1D—Quantification of results from C. ***p<0.001 (1-way Anova). FIG. 1E—MCF10A cells treated with EGF or DEX were followed using time-lapse microscopy. Shown are rose plots of single-cell trajectories; red tracks indicate migration persistence smaller than 0.3. FIG. 1F—Quantification of migration parameters from E (means±SEM, from 60 cells). FIG. 1G—Wound closure assays were performed following the indicated treatments of MCF10A cells. Green lines mark migration fronts. FIG. 1H—Quantification of time-lapse movies from FIG. 1G. Five-minute frames were used (fine lines) and both average migration distance and velocity are presented. FIG. 1I—MCF10A cells (5×10⁵ cells/well) were plated in Transwell chambers and treated with the following agents, either alone or in combinations: EGF (10 ng/ml), DEX (100 nM), estradiol (E2; 30 nM), progesterone (PRG; 30 nM) or medroxyprogesterone acetate (MPA; 100 nM). Shown are representative images of the lower sides of triplicate 8 μm filters, which were stained with crystal violet 20 hours later. The experiment was repeated thrice. FIG. 1J—MCF10A cells pre-treated for 24 hours with of EGF, DEX or the combination. Thereafter, cells were stained for the apoptosis marker annexin V and the necrosis marker propidium iodide (PI), and later assayed using flow cytometry. FIG. 1K—MCF10A cells were transfected with control siRNA oligonucleotides, or with NR3C1-(GR) specific siRNAs, and 48 hours later whole cell extracts were probed for either GR or tubulin. FIG. 1L—MCF10A cells were treated for either 5 or 10 minutes with EGF (10 ng/ml) or DEX (100 nM). Thereafter, cell extracts were fractionated into nuclear and cytoplasmic fractions prior to immunoblotting with antibodies to GR, lamin B or the heat shock protein 90 (HSP90). FIG. 1M—MCF10A cells were treated for 30 minutes with DEX (100 nM). Paraformaldehyde-fixed cells were permeabilized and incubated overnight with a GR-specific antibody (green) and with DAPI (blue). Bars, 50 p.m.

FIGS. 2A-E show that activated GRs repress EGF-induced transcriptional programs. FIG. 2A—RNA was isolated from MCF10A cells pre-treated as indicated, and hybridized to Affymetrix Exon Arrays. The heatmaps display RNA fold changes, which were clustered into four groups and ordered according to RNA's peak time. FIG. 2B—A scheme depicting relationships among EGFR, GR and the four modules. FIG. 2C—For each time point, we calculated the average gene expression fold changes (combined treatment minus ‘EGF only’ treatment), and then presented the resulting average relative to t=240 min. FIG. 2D—The average difference between the fold change following EGF treatment or ‘DEX plus EGF’ treatment was used to present the extent of repression relative to t=40 min. FIG. 2E—GR signalling regulates EGF-induced transcriptional programs. MCF10A mammary epithelial cells were stimulated with EGF for the indicated time intervals and RNA samples were processed for high throughput gene expression analyses using real time PCR and microfluidic dynamic arrays (Fluidigm® Real-Time PCR). Both mRNA and pre-mRNA levels were surveyed using specific oligonucleotides. Genes are arranged according to the peak time of the respective mRNA levels.

FIGS. 3A-I show that GR enhances expression of negative feedback regulators of EGFR signalling. FIG. 3A—Serum-starved MCF10A cells were treated with EGF or DEX. qPCR analysis was performed using RNA and primers corresponding to pre-mRNAs (dashed lines) or the mature forms (solid lines). FIG. 3B—A scheme depicting negative feedback regulators of EGFR signalling. FIGS. 3C-D—Cells were stimulated as in A and extracts were immunoblotted for ERRFI1, GR and ERK2. Normalized ERRFI1 signals are shown. FIGS. 3E-F—Active ERK signals (pERK) were determined, normalized and presented. FIG. 3G—MCF10A derivatives stably expressing ERRFI1-specific shRNAs were tested for migration following the indicated treatments. The results were analysed as in FIG. 1D. *p<0.05; ***p<0.0001 (one-way Anova). FIG. 3H—Serum-starved MCF10A cells were pre-incubated for 20 minutes with actinomycin D (1 μg/m1), and thereafter stimulated for the indicated time intervals with EGF or DEX. This was followed by preparation of cell extracts and immunoblotting with an antibody to active (phosphorylated) ERK. FIG. 3I—The pERK signals from FIG. 3H and additional experiments were quantified, normalized to total ERK2 levels and presented.

FIGS. 4A-G show that GR rewires EGF-induced transcriptional programs through IR nGREs and transrepression. FIG. 4A—MCF10A cells were analysed for expression of the indicated genes as in FIG. 3A. FIG. 4B—Cells were treated for 4 hours, as indicated, and extracts were tested for HB-EGF using ELISA. Results represent biological duplicates performed in technical triplicates. FIG. 4C—Pscan (159dot149dot160dot51/pscan/; Jaspar database) was used to find over-represented TF binding sites in EGF-inducible Module B genes (n=593). The Bonferroni corrected p-values for multiple testing are shown. In addition, the set of genes was analysed using the Cscan compendium of chromatin immunoprecipitation-sequencing (ChIP-Seq) experiments, and the respective p-values presented as the median of Bonferroni corrected values. O/E, observed relative to expected. FIG. 4D—The indicated siRNAs were transfected into MCF10A cells, which were re-seeded 48 hours later, scratched and stimulated with EGF. Migration (average±SEM) was assayed in triplicates. FIG. 4E—Hypergeometric distribution of MCF10A expressed genes, including IR nGRE-containing, DEX-downregulated genes and Module B genes. Overlapping genes are listed; p=1.28×10⁻⁶. FIG. 4F—Two previously breast cancer clinical datasets were analyzed for relapse-free survival (RFS; see main text). Tumors were stratified according to high (red) or low (blue) expression of the NR3C1 (GR) gene. Patient numbers and p-values are indicated. FIG. 4G—Patients included in the Ivshina dataset of breast cancer were stratified according to the Elston (NGS) histologic grade, whereby score 1 is the best and 3 is the worst. Note that low GR expression levels associate with shorter survival rates in patients of grades 2 and 3. The expression level of GR was detected in each histological group, and it appears to be lower in grade 2 and 3, relative to grade 1. p=0.0014 (Anova).

FIGS. 5A-D show a diurnal control of EGFR transcriptional programs in animals. FIGS. 5A-B—Mouse livers (n=4) were collected at the indicated time of the day or night (grey areas), and analysed using RT-PCR for ERRFI1 and DUSP1 (negative regulators) or HBEGF and TGFA (positive regulators). Zeitgeber (ZT) zero indicates light ON. FIG. 5C—Serum from wild type mice was collected at ZT4 and ZT10 (“day”) or ZT15 and ZT20 (“night”) and assayed using ELISA for TGFA and HBEGF. FIG. 5D—Composite panel of experimentally determined antithetical oscillations of EGFR's negative (Mig6, Dusp1, Sulf1) and positive feedback regulators (Tgfa, Hbegf, Ereg) as reported in the Circa DB gene expression database (bioinfdotitmat.upenndotedu/circa/query). The following murine tissues were used as sources of RNA during the active and resting phases: liver, pituitary, brain stem and brown adipose (48 hour Hughes 2009, Affymetrix).

FIGS. 6A-F show that circadian oscillations of corticosteroids control negative feedback of EGFR in animals and might affect tumor growth. FIG. 6A—WT and CRFR1^{-/-} (KO) mice were sacrificed at the indicated times and liver mRNA was extracted. Errf1 and Dusp1 were assayed using RT-PCR. FIG. 6B—The status of ERK activation in WT and CRFR1^{-/-} (KO) mice was determined using immunoblotting of liver extracts. FIG. 6C—The normalized level of ERK activity is plotted, along with the corresponding corticosteroid serum concentration (ng/ml) as detected by using a radioimmunoassay (dashed lines). A indicates the lowest point of ERK activity corresponding to the peak of GCs in WT mice. Note that this pattern is lost in CRFR1^{-/-} (KO) mice. FIGS. 6D-F—CD1/nude mice were injected subcutaneously with 5×10⁶ N87 cells. Lapatinib treatment (40 mg/kg/day) was started once tumors became palpable, about 2 weeks after the inoculation. The “day” group received the Lapatinib by oral gavage just before the beginning of the resting phase, while the night group received oral gavage Lapatinib at the beginning of the active phase (see a scheme). Tumor sizes±SEM are presented. In the end of the experiment tumors were weighted (each dot represents one animal) and photographed.

FIGS. 7A-D show that high GR abundance associates with better prognosis of breast cancer patients. FIG. 7A—Breast cancer specimens from the METABRIC dataset were classified into two equal size groups according to GR transcript levels. The respective relapse-free survival (RFS) of each group is shown. FIG. 7B—Breast cancer patients were divided into three groups according to tumor stage, and patient survival was analysed relative to GR abundance. FIG. 7C—Shown are representative sections of GR immunostaining of invasive breast carcinomas (331 patients). The fraction of pERK-positive specimens in each group was determined (p=0.013; Chi-square test). FIG. 7D—A model depicting the crosstalk between EGFR and GR during the active phase (right; high GC level) and the resting phase (night; low GC). Both positive and negative feedback loops regulating EGFR signalling are indicated, and signalling is divided into three layers.

### DESCRIPTION OF SPECIFIC EMBODIMENTS OF THE INVENTION

The present invention, in some embodiments thereof, relates to compositions and methods for treating cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst searching for novel therapeutic modalities for the treatment of cancer, the present inventors have observed that a steroid hormone, glucocorticoid, inhibits signalling downstream to the receptor tyrosine kinase (RTK), EGFR. Without being bound by theory, it is suggested that glucocorticoid signalling suppresses EGFR's positive feedback loops, mainly production of auto-stimulatory EGFR ligands, and simultaneously triggers negative feedback loops that normally restrain EGFR. Animal studies revealed that by altering EGFR' s feedback, glucocorticoids regulate signalling in a circadian manner. Therefore, whilst further conceiving the present invention, the present inventors have shown in mice that EGFR signals are suppressed by high glucocorticoids during the active phase of the day, but they are active during the resting phase. Consistent with this model, treatment of animals bearing EGFR-driven tumors with an EGFR-specific drug is more effective if administered during the resting phase of the day. These findings offer a new, circadian clock-based paradigm in cancer therapy.

Thus, according to an aspect of the invention there is provided a method of treating cancer in a subject in need thereof, the method comprising administering to the subject a receptor tyrosine kinase (RTK)-specific cancer therapy and a glucocorticoid or a glucocorticoid analog, such that an efficacy window of the RTK-specific cancer therapy and an efficacy window of the glucocorticoid or glucocorticoid analog substantially overlap.

As used herein the term “cancer” relates to a malignant tumor which expresses a receptor tyrosine kinase (RTK), e.g., an ErbB family member, e.g., EGFR and in which expression of the RTK is associated with onset or progression of the disease. Alternatively, the cancer contemplated herein is where the RTK specific cancer therapy is putatively helpful.

As used herein “an RTK” refers to the cell surface bound form of a protein tyrosine kinase (E.C. 2.7.1.112, 2.7.10.1). Surface expression/activation of the RTK is typically associated with the onset or progression of a disease, usually a malignant disease, such as cancer.

According to a specific embodiment, the cells of the cancer express the RTK.

According to a specific embodiment, the cells of the cancer express the RTK (i.e., mRNA and/or protein) at a higher level as compared to same in cells of a non-malignant tissue of the same developmental stage.

According to a specific embodiment, the cells of the cancer exhibit genetic amplification in the RTK locus.

According to an alternative or additional specific embodiment, the cells of the cancer display activation of the RTK. According to an embodiment of the invention, the cells express a mutant form of the RTK, which renders its signalling ligand-independent (i.e., constitutively active). According to an embodiment of the invention, the tumor expresses a constitutively active ErbB protein e.g., a 4(2-7) EGFR, a mutant form of EGFR specifically expressed in glioblastoma.

Methods of determining RTK expression and activation include but are not limited to immune-staining, Western blot analysis, immunoprecipitation and various kinase assays e.g., in vitro kinase assays.

Non-limiting examples of RTKs according to some embodiments of the invention include, but are not limited to, AATK; AATYK; AATYK2; AATYK3; ACH; ALK; anaplastic lymphoma kinase; ARK; ATP:protein-tyrosine O-phosphotransferase; AXL; Bek; Bfgfr; BRT; Bsk; C-FMS; CAK; CCK4; CD115; CD135; CDw135; Cekl; Cek10; Cek11; Cek2; Cek3; Cek5; Cek6Cek7; CFD1; CKIT; CSF1R; DAlk; DDR1; DDR2; Dek; DKFZp434C1418; Drosophila Eph kinase; DRT; DTK; Ebk; ECK; EDDR1; Eek; EGFR; Ehk2; Ehk3; Elk; EPH; EPHA1; EPHA2; EPHA6; EPHA7; EPHA8; EPHB1; EPHB2; EPHB3; EPHB4; EphB5; ephrin-B3 receptor tyrosine kinase; EPHT; EPHT2; EPHT3; EPHX; ERBB; ERBB1; ERBB2; ERBB3; ERBB4; ERK; Eyk; FGR1; FGFR2; FGFR3; FGFR4; FLG; FLK1; FLK2; FLT1; FLT2; FLT3; FLT4; FMS; Fv2; HBGFRHEK11; HEK2; HEK3; HEK5; HEK6; HEP; HER2; HER3; HER4; HGFR; HSCR1; HTK; IGF1R; INR; INSRR; insulin receptor protein-tyrosine kinase; IR; IRR; JTK12; JTK13; JTK14; JWS; K-SAM; KDR; KGFR; KIA0641; KIAA1079; KIAA1459; Kil; Kin15; Kin16; KIT; KLG; LTK; MCF3; Mdkl; Mdk2; Mdk5; MEhk1; MEN2A/B; Mep; MER; MERTK; MET; Mlk1; Mlk2; Mrk; MSTR; MTC1; MUSK; Mykl; N-SAM; NEP; NET; Neu; neurite outgrowth regulating kinase; NGL; NOK; nork;; Nsk2; NTRK1; NTRK2; NTRK3; NTRK4; NTRKR1; NTRKR2; NTRKR3; Nuk; NYK; PCLPDGFR; PDGFRA; PDGFRB; PHB6;; RET; RON; ROR1; ROR2; ROS1; RSE; RTK; RYK; SEA; Sek2; Sek3; Sek4; Sfr; SKY; STK; STK1; TEK; TE; TIE1; TIE2; TIF; TKT; TRK; TRKA; TRKB; TRKC; TRKE; TYK1; TYRO10; Tyroll; TYRO3; Tyro5; Tyro6; TYRO7; UFO; VEGFR1; VEGFR2; VEGFR3; Vik; YK1; Yrk.

Specific examples of RTKs which can be used in accordance with this aspect of the present invention are listed in Table 1 below.

TABLE 1

| | | Accession | Examples of | | |
|---|---|---|---|---|---|
| | | number/SEQ | associated | | RTK |
| Full name | Reference | ID NOs: | Pathologies | RTK | subfamily |
| epidermal | Silvestri G A and | NP_958441/ | non-small cell | EGFR/ErbB- | ErbB |
| growth | Rivera M P, | 108 | lung cancer | 1/HER1 | subfamily |
| factor | Chest. | | | | |
| receptor | 128(6): 3975-84, | | | | |
| | 2005. | | | | |
| | Snyder L C, et | | colorectal cancer | | |
| | al., Clin | | head and neck | | |
| | Colorectal | | cancer | | |
| | Cancer. 1 2: S71-80, | | | | |
| | 2005. | | | | |
| | Slamon D J, et | Sprot: | breast ovarian and | ErbB- | ErbB |
| | al,. Science 244: | P04626/109 | lung cancer | 2/HER2 | subfamily |
| | 707-712, 1989. | | transitional cell | | |
| | Visakorpi T, et | | carcinoma of the | | |
| | al., Clin. Cancer | | bladder | | |
| | Res. 9 (14), | | | | |
| | 5346-5357 | | | | |
| | (2003) | | | | |
| | Huynh H, et al., | | prostate cancer | | |
| | Int. J. Oncol. 23 | | | | |
| | (3), 821-829 | | | | |
| | (2003) | | | | |
| Tyrosine | van der Horst | NP_001005915/ | breast cancer | ErbB-3/ | ErbB |
| kinase- | E H, et al., Int. J. | 110 | | HER3 | subfamily |
| type cell | Cancer 115 (4): | | | | |
| surface | 519-527, 2005 | | | | |
| receptor | Visakorpi T, et | | transitional cell | | |
| HER3 | al., Clin. Cancer | | carcinoma of the | | |
| | Res. 9 (14): | | bladder | | |
| | 5346-5357, | | | | |
| | 2003 | | | | |
| | Huynh, H., et al., | | prostate cancer | | |
| | Int. J. Oncol. 23 | | | | |
| | (3), 821-829 | | | | |
| | (2003) | | | | |
| | Kobayashi, M., | | adenocarcinoma | | |
| | et al., Oncogene | | | | |
| | 22 (9), 1294-1301 | | | | |
| | (2003) | | | | |
| | de Vicente et al., | | oral squamous | | |
| | Med Oral. | | cell carcinoma | | |
| | 8(5): 374-81, | | | | |
| | 2003 | | | | |
| | de Vicente et al., | Q15303/111 | oral squamous | ErbB-4/ | ErbB |
| | Med Oral. | | cell carcinoma | HER4 | subfamily |
| | 8(5): 374-81, | | | | |
| | 2003 | | | | |
| | Merimsky O., et | | bone sarcoma | | |
| | al., Oncol Rep. | | | | |
| | 10(5): 1593-9, | | | | |
| | 2003 | | | | |
| platelet- | Matsuda, M., et | Sprot: | glomerulonephritis | PDGFR | platelet- |
| derived | al, J. Neural | P16234/112 | (non cancer) | alpha | derived |
| growth | Transm. 17 (1), | | | | growth |
| factor | 25-31, 1997 | | | | factor |
| receptor | Wilczynski, S P. | | epithelial ovarian | | receptor |
| alpha | et al., Hum. | | cancers | | subfamily |
| | Pathol. 36 (3), | | | | |
| | 242-249, 2005 | | | | |
| | Ebert, M., et al., | | human pancreatic | | |
| | Int. J. Cancer 62 | | cancer | | |
| | (5), 529-535, | | | | |
| | 1995 | | | | |
| platelet- | Tamborini E, et | NP_002600 | synovial sarcoma | PDGFR | platelet- |
| derived | al., Clin. Cancer | (precursor)/113 | | beta | derived |
| growth | Res. 10 (3): | | | | growth |
| factor | 938-943, 2004 | | | | factor |
| receptor | Matsuda M, et | | glomerulonephritis | | receptor |
| beta | al., J. Neural | | (non cancer) | | subfamily |
| | Transm. 17 (1): | | | | |
| | 25-31, 1997 | | | | |
| | Wilczynski S P,. | | epithelial ovarian | | |
| | et al., Hum. | | cancers | | |
| | Pathol. 36 (3): | | | | |
| | 242-249, 2005 | | | | |
| | Ebert M, et al., | | pancreatic | | |
| | Int. J. Cancer 62 | | cancer | | |
| | (5): 529-535, | | | | |
| | 1995 | | | | |
| vascular | Longatto F A, et | NP_891555/ | breast cancer | Flt-4/ | platelet- |
| endothelial | al., Pathol Res | 114 | | VEGFR-3 | derived |
| growth | Pract.; 201(2): 93-9, | | | | growth |
| factor | 2005 | | | | factor |
| receptor | Kojima H, et al., | | lung | | receptor |
| | Cancer 104 (8): | | adenocarcinoma | | subfamily |
| | 1668-1677, | | | | |
| | 2005 | | | | |
| fms- | Schmidt-Arras | NP_004110/ | hematologic | Flt-3 | platelet- |
| related | D, et al., Curr | 115 | malignancies: | | derived |
| tyrosine | Pharm. | | acute myeloid | | growth |
| kinase 3/ | 10(16): 1867-83, | | leukemia | | factor |
| Vascular | 2004 | | | | receptor |
| endothelial | Van Vlierberghe | | pediatric T-cell | | subfamily |
| growth | P., et al., Blood | | acute | | |
| factor | 106 (13): 4414-4415, | | lymphoblastic | | |
| receptor 3 | 2005 | | leukemias | | |
| hepatocyte | Dietrich S, et | NP_000236 | upper | c-MET/ | hepatocyte |
| growth | al., J. Environ. | (Precursor)/116 | aerodigestive | HGFR | growth |
| factor | Pathol. Toxicol. | | malignancies | | factor |
| receptor | Oncol. | | | | receptor |
| | 24(3): 149-62, | | | | subfamily |
| | 2005. | | | | |
| ephrin | Ireton R C and | NP_004422 | breast, prostate, | EphA2/Eck | ephrin |
| receptor | Chen J,: Curr. | (Precursor)/117 | lung, and colon | | receptor |
| EphA2 | Cancer Drug | | cancers | | family |
| | Targets. | | | | |
| | (3): 149-57, | | | | |
| | 2005 | | | | |
| ephrin | Xia G, et al., | NP_004435 | prostate cancer | EphB4 | ephrin |
| receptor | Cancer Res. 65 | (Precursor)/118 | | | receptor |
| EphB4 | (11): 4623-4632, | | | | family |
| | 2005 | | | | |
| | Malavaud, B., | NP_056934 | stem cell | FGFR1 | fibroblast |
| | Oncogene 23 | (precursor)/119 | leukemia | | growth |
| | (40): 6769-6778, | | lymphoma | | factor |
| | 2004 | | syndrome (SCLL) | | receptor |
| | Kranenburg, A. | | bladder | | family |
| | et al., R. Am. J. | | carcinoma | | |
| | Respir. Cell | | chronic | | |
| | Mol. Biol. 27 | | obstructive | | |
| | (5): 517-525, | | pulmonary | | |
| | 2002 | | disease | | |
| | | | (non cancer) | | |
| keratinocyte | de Ravel T J, et | NP_075265 | Crouzon | KGFR/FGFR2 | fibroblast |
| growth | al., Eur. J. | precursor)/120 | syndrome (non | | growth |
| factor | Hum. Genet. 13 | | cancer) | | factor |
| receptor | (4), 503-505, | | | | receptor |
| | 2005 | | | | family |
| | Jang J H, et al., | | gastric and | | |
| | Cancer Res. 61 | | colorectal cancers | | |
| | (9), 3541-3543 | | | | |
| | (2001) | | | | |
| | Kurban G, et al., | | uterine cervical | | |
| | Oncol. Rep. 11 | | cancer | | |
| | (5): 987-991, | | | | |
| | 2004 | | | | |
| fibroblast | L'Hote C G, and | NP_075254 | multiple | FGFR3 | fibroblast |
| growth | Knowles M A | (precursor)/121 | myeloma, | | growth |
| factor | Exp. Cell Res. | | cervical | | factor |
| receptor 3 | 304(2): 417-31, | | carcinoma and | | receptor |
| | 2005 | | carcinoma of the | | family |
| | | | bladder | | |
| Epithelial | Matsuyama W, | NP_054699/ | pulmonary | DDR1 | Insulin |
| discoidin | et al., Am. J. | 122 | sarcoidosis | | receptor |
| domain | Respir. Cell | | (non cancer) | | subfamily |
| receptor 1 | Mol. Biol. 33 | | | | |
| | (6): 565-573, | | | | |
| | 2005 | | | | |
| | Heinzelmann- | | breast, ovarian, | | |
| | Schwarz V A, et | | esophageal, and | | |
| | al., Clin. Cancer | | pediatric brain | | |
| | Res. 10 | | tumors | | |
| | (13): 4427-4436, | | | | |
| | 2004 | | | | |
| insulin- | Knowlden J M, | NP_000866 | breast cancer | IGF1R | Insulin |
| like | et al., | (precursor)/123 | | | receptor |
| growth | Endocrinology | | | | subfamily |
| factor 1 | 146 (11): 4609-4618, | | | | |
| receptor | 2005 | | | | |
| Proto- | Gal, A., Nat. | Q12866 | retinitis | MERTK | Axl/Ufo |
| oncogene | Genet. 26 (3), | (precursor)/124 | pigmentosa (non | | subfamily |
| tyrosine- | 270-271 (2000) | | cancer) | | |
| protein | | | | | |
| kinase | | | | | |
| MER | | | | | |
| AXL | Chung B I, et al., | NP_001690/ | renal cell | Axl/Ufo | Axl/Ufo |
| receptor | DNA Cell Biol. | 125 | carcinoma | | subfamily |
| tyrosine | 22 (8): 533-540, | | | | |
| kinase | 2003 | | | | |
| | Ito M, Thyroid | | pediatric thyroid | | |
| | 12 (11), 971-975, | | carcinomas | | |
| | 2002 | | | | |
| | Sun W S, et al., | | ovarian | | |
| | Mol. Hum. | | endometriosis | | |
| | Reprod. 8 (6): | | (non cancer) | | |
| | 552-558 2002 | | | | |
| | O'Bryan J. P., | | human myeloid | | |
| | Mol. Cell. Biol. | | leukemia | | |
| | 11: 5016-5031 | | | | |
| | (1991). | | | | |

According to a specific embodiment, the RTK belongs to the ErbB family.

The ErbB family of polypeptides relates to the group of four structurally related receptor tyrosine kinases, which in humans includes HER1 (EGFR, ErbB 1), HER2 (Neu, ErbB2), HER3 (ErbB3), and HER4 (ErbB4).

As used herein “EGFR” refers to a receptor tyrosine kinase (RTK) of the epidermal growth factor receptor family, EGFR_HUMAN, P00533, also referred to as HER1, mENA and ErbB-1.

As used herein “ErbB-2” refers to a receptor tyrosine kinase (RTK) of the epidermal growth factor receptor family, ERBB2_HUMAN, P04626, also referred to as HER2, NEU and p185erbB-2.

As used herein “ErbB-3” refers to a receptor tyrosine kinase (RTK) of the epidermal growth factor receptor family, also referred to as HER3.

According to an embodiment of the invention the cancer is a solid tumor.

According to an embodiment of the invention the cancer is a non-solid tumor.

According to an embodiment of the invention the cancer is a primary tumor.

According to an embodiment of the invention the cancer is a metastatic tumor.

According to an embodiment of the invention the cancer is a recurrent tumor.

According to an embodiment of the invention the cancer is chemotherapy resistant.

Examples of cancer types which can be treated according to some embodiments of the invention, include, but are not limited to, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central

Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

An exemplary list of cancers which can be treated according to some embodiments of the invention, include advanced and non-advanced cancers including metastasized cancers such as metastatic and non-metastatic lung cancer, breast cancer, head and neck cancer, (HNSCC), pancreatic cancer, pharyngeal cancer, colorectal cancer, anal cancer, glioblastoma multiforme, epithelial cancers, renal cell carcinomas, acute or chronic myelogenous leukemia and other leukemias.

According to specific embodiments, the treated cancer (e.g., ErbB expressing cancer, e.g., EGFR or HER2) is a lung cancer such as a non-small lung cancer e.g., squamous cell carcinoma, large cell carcinoma or adenocarcinoma or a small cell lung cancer such as small cell carcinoma (oat cell cancer) or combined small cell carcinoma. In a particular embodiment the treated lung cancer comprises squamous cell carcinoma.

However, as noted above any cancer wherein the RTK-specific cancer therapies are potentially useful is contemplated such as advanced or non-advanced, non-metastatic and metastatic forms of colorectal cancer, pancreatic cancer, breast cancer, head and neck cancer, esophageal cancer, lung cancer, oval an cancer, cervical cancer, renal cancer, prostate cancer, testicular cancer, brain cancer, and others.

According to a specific embodiment, when targeting EGFR or ErbB-2, examples of cancers include, but are not limited to, carcinoma, adenocarcinoma, lung cancer, liver cancer, colorectal cancer, brain, head and neck cancer (e.g., neuro/glioblastoma), breast cancer, ovarian cancer, transitional cell carcinoma of the bladder, prostate cancer, oral squamous cell carcinoma, bone sarcoma, biliary tract cancer such as gallbladder carcinoma (GBC), kidney cancer and pancreatic cancer.

According to a specific embodiment the cancer is pancreatic cancer.

As used herein “pancreatic cancer” refers to pancreatic adenocarcinomas, adenosquamous carcinomas, signet ring cell carcinomas, hepatoid carcinomas, colloid carcinomas, undifferentiated carcinomas, and undifferentiated carcinomas with osteoclast-like giant cells.

According to a specific embodiment, the cancer is not lymphoma, prostate cancer or breast cancer.

As used herein “a receptor tyrosine kinase (RTK)-specific cancer therapy” refers to a molecule which at least partially suppresses an RTK signalling (ligand-induced or constitutive signalling) as compared to said signalling under the same conditions (e.g., same cell or cell type) however in the absence of the molecule. RTK signalling can be assayed using methods which are well known in the art including, but not limited to, in-vitro kinase assay, receptor autophysphorylation assay, down-stream signalling (e.g., by co-immunoprecipitation), cell proliferation (e.g., MTT or thymidine incorporation assay) and receptor endocytosis. Non-limiting examples of such molecules include, but are not limited to, small molecule tyrosine kinase inhibitors, antagonistic antibodies, peptide antagonists, aptamers, and ligand sinks. Following is a further description of some of these modalities.

Small molecule tyrosine kinase inhibitors—Small molecule tyrosine kinase inhibitors (TKIs) target the ATP binding pocket of RTKs. TKIs antagonize RTK coupling to biological responses by inhibiting RTK tyrosine kinase activity and phosphorylation-dependent RTK coupling to signalling effectors. Examples of such molecules include, but are not limited to, the Abl/c-Kit TKI imatinib (Gleevec®—Novartis), gefitinib (Iressa™—Astra-Zeneca) and erlotinib (Tarceva®—Genentech).

Antibodies—monoclonal antibodies that target extracellular epitopes of cell surface proteins whose expression is associated with a pathologic state. In some cases these antibodies appear to function primarily by eliciting an immune response specific for the cells that express the RTK. Alternatively, antibodies act as ligand sinks, inhibitors of ligand binding, inhibitors of receptor dimerization, and agents with other mechanisms of action.

Ligand sinks—Ligand sinks antagonize RTK signalling by binding the RTK agonist and preventing the agonist from binding to the RTK and stimulating its signalling. One example is the monoclonal antibody bevacizumab (Avastin®—Genentech), which binds to vascular endothelial growth factor (VEGF). This prevents VEGF from binding to the VEGF receptor and prevents VEGF stimulation of VEGF receptor signalling.

Inhibitors of ligand binding—Other monoclonal antibodies bind to an RTK and prevent agonist binding to the RTK and agonist stimulation of RTK signalling. Theoretically, a variety of mechanisms of action are possible. Monoclonal antibodies could directly compete with agonists for binding to a common or overlapping binding site on the RTK. Cetuximab (Erbitux®—Bristol-Myers Squibb) is an example of this class of agents; it competes with EGF and other EGFR agonists for binding to EGFR, thereby inhibiting agonist-induced EGFR signalling. Alternatively, monoclonal antibodies can inhibit agonist-induced RTK signalling by inducing the RTK to adopt a conformation with lower affinity for agonist (allosteric inhibition). Alternatively, monoclonal antibodies can inhibit agonist-induced RTK signalling by inducing the RTK to internalize thus being less available for agonist binding.

Inhibitors of receptor dimerization—As many RTKs act through dimerization or heterodimerization, the inhibitor may interfere with this stage of signalling. Pertuzumab (fka Omnitarg) is an antibody specific for ErbB2 (HER2/Ncu) RTK that inhibits ErbB2 heterodimerization with other ErbB family receptors, including EGFR and ErbB3 (HER3). Because ErbB2 lacks a specific soluble agonist, agonist binding to an ErbB receptor other than ErbB2 and consequent heterodimerization and cross-talk with ErbB2 is a common mechanism by which ErbB2 signalling can be regulated.

Other mechanisms of action—Trastuzumab (Herceptin®) is specific for ErbB2 and is used to target tumors that overexpress ErbB2. A number of mechanisms, including antibody-dependent cellular cytotoxicity, may account for the antitumor activities of trastuzumab. However, 4D5, the mouse monoclonal antibody from which trastuzumab is derived, stimulates ErbB2 tyrosine phosphorylation and internalization. This mechanism may also account for some of the antitumor activities displayed by trastuzumab and other antibodies.

Other agents—RTK fragments that include the agonist-binding domain(s) may serve as decoy receptors for agonists (agonist sinks). For example, a recombinant soluble protein containing the extracellular subdomains I-III of ErbB4 antagonizes agonist-induced signalling by ErbB4. Proteins that are not derived from RTKs may also function as agonist sinks. Perhaps the best know is the drosophila Argos protein, which binds to the drosophila EGF homolog Spitz and antagonizes stimulation of drosophila EGFR (DER) signalling by preventing Spitz binding to DER. Finally, a fragment of an RTK agonist that retains the site of binding to the RTK may competitively antagonize agonist-induced signalling by that RTK. For example, a fragment corresponding to residues 33-42 of murine EGF inhibits EGF stimulation of endothelial cell motility and EGF stimulation of chicken egg angiogenesis. Table 2 lists some FDA approved RTK inhibitors.

TABLE 2
FDA-Approved EGFR Inhibitors

| | | Initial |
|---|---|---|
| Drug | | Approval |
| (Trade name) | Class Target | Date |
| Cetuximab | mAb EGFR | February 2004 |
| (Erbitux) | | |
| ImClone, | | |
| Bristol- | | |
| Myers Squibb | | |
| Erlotinib | TKI EGFR | November 2004 |
| (Tarceva) | | |
| OSI | | |
| Pharmaceuticals | | |
| Gefitinib | TKI EGFR | May 2003 |
| (Iressa) | | |
| AstraZeneca | | |
| Lapatinib | TKI EGFR/HER2 | March 2007 |
| (Tykerb) | | |
| SmithKline | | |
| Beecham | | |
| Panitumumab | mAb EGFR | September 2006 |
| (Vectibix) | | |
| Amgen | | |

TKI = tyrosine kinase inhibitor;
mAb = monoclonal antibody;
NSCLC = non-small-cell lung cancer;
HNSCC = squamous cell carcinoma of the head and neck

According to some embodiments of the invention, EGFR inhibitors include, but are not limited to Sunitinib or Sutent (N-(2-diethylaminoethyl)-5-[(Z)-(5-fluoro-2-oxo-1H-indol -3-ylidene)methyl-]-2,4-dimethyl-1H-pyrrole-3-carboxamide) marketed by Pfizer, Gefitinib or N-(3-chloro-4-fluoro-phenyl)-7-methoxy-6-(3-morpholin-4-ylpropoxy)quinazo-lin-4-amine marketed by Astra7eneca, and Zalutumumab in clinical development by GenMab.

Examples of HER2 inhibitors include, but are not limited to Herceptin™ (trastuzumab), Tykerb™ (Lapatinib), Kadeyla™ (ado-trastuzumab emtansine) and Prejeta™ (pertuzumab).

According to some embodiments of the invention, the tyrosine kinase inhibitors include, but are not limited to, Axitinib (Inlyta), Dasatinib (Sprycel), Erlotinib (Tarceva), Nilotinib (Tasigna), Pazopanib (Votrient) and Sorafenib (Nexavar).

The term “antibody” as used in this invention includes intact molecules as well as functional fragments thereof, such as Fab, F(ab')2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab', the fragment of an antibody molecule that can be obtained by treating whole antibody with pepsin, followed by reduction, to yield an intact light chain and a portion of the heavy chain; two Fab' fragments are obtained per antibody molecule; (3) (Fab')2, the fragment of the antibody that can be obtained by treating whole antibody with the enzyme pepsin without subsequent reduction; F(ab')2 is a dimer of two Fab' fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

According to an embodiment of the invention, when the RTK-specific cancer therapy is directed against an ErbB molecule, the inhibitor is selected from the group consisting of Erlotinib, Genfitinib and Lapatinib.

Alternatively, according to an embodiment of the invention, the RTK-specific cancer therapy is selected from the group consisting of Panitumumab and Cetuximab.

As used herein the term “glucocorticoid” or “glucocorticoid analog” or as abbreviated herein “GC” refers to a naturally occurring or synthetic molecule that binds and activates the glucocorticoid receptor (GR) also known as NR3C1 (nuclear receptor subfamily 3, group C, member 1).

According to a specific embodiment the “glucocorticoid analog” is non-steroidal.

According to a specific embodiment the “glucocorticoid analog” is steroidal.

According to a specific embodiment, the glucocorticoid is a physiological molecule, i.e., naturally occurring (e.g., cortisol).

Generally, any corticosteroid, e.g., glucocorticoid, can be used in the methods or combinations provided herein. Exemplary glucocorticoids include, but are not limited to: alclometasones, algestones, beclomethasones (e.g. beclomethasone dipropionate), betamethasones (e.g. betamethasone 17-valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones and hydrocortisones (e.g. hydrocortisone acetate), cortivazols, deflazacorts, desonides, desoximetasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, fluprednisolones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylprednisolones (methylprednisolone aceponate, methylprednisolone acetate, methylprednisolone hemisuccinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemisuccinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21-palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980).

In some examples, the glucocorticoid is selected from among cortisones, dexamethasones, hydrocortisones, methylprednisolones, prednisolones and prednisones. In a particular example, the glucocorticoid is dexamethasone.

Examples of non-steroidal analogs, according to some embodiments of the invention, include, but are not limited to, CpdA, LGD5552, AL-438, ZK245186, Quinol-4-ones, ZK216348 and BI115.

According to a specific embodiment the RTK inhibitor is used together with a non-steroidal GR analog.

As used herein the term “subject” or “subject in need thereof” refers to an individual who has been diagnosed with cancer, as described herein. According to a specific embodiment, the subject is a human subject. According to a specific embodiment, the subject is a female subject. According to a specific embodiment, the subject is a male subject. The subject may be at any age (e.g., new-born, infant, child, adolescent, adult, or of the elderly population, according to FDA classification groups). According to a specific embodiment, the subject suffers from metastatic cancer or a locally advanced disease.

The term “treating” refers to inhibiting, preventing or arresting the development of cancer and/or causing the reduction, remission, or regression of a cancer. Those of skill in the art will understand that various methodologies and assays can be used to assess the development of cancer, and similarly, various methodologies and assays may be used to assess the reduction, remission or regression of the cancer.

According to a specific embodiment, the methods described herein can be used for the prevention of cancer. As used herein, the term “preventing” refers to keeping a disease, disorder or condition from occurring in a subject who may be at risk for the disease, but has not yet been diagnosed as having the disease.

As used herein, the phrase “efficacy window” describes a time frame during which an active agent exhibits a desired pharmacological effect, herein an RTK inhibition effect or a glucocorticoid receptor activation effect, upon administration. In other words, this phrase describes that time period at which the plasma concentration of an active agent is equal to or higher than a minimal pharmacologically effective concentration thereof.

As is well known in the art, an efficacy window of an agent depends on various factors such as systemic absorbance rate, the time required to reach a plasma peak concentration and/or clearance rate.

As described hereinabove, since GCs activity is circadianly regulated, it is better to administer the RTK inhibitor during the day i.e., when the endogenous GC signalling is active, or at the resting phase (i.e., night, e.g., when cortisol levels drop) while augmenting the treatment with exogenously administered GC or analog thereof. Accordingly, administration of the RTK inhibitor and/or GC (or analog) is under a circadian regimen. Thus for example, the RTK inhibitor may be administered at the beginning of the active phase (day). Alternatively or additionally, the RTK inhibitor is administered during the night but in conjunction with GC. Yet alternatively, RTK inhibitor is administered to achieve an efficacy window which overlaps that of exogenously administered GC.

Methods of determining the circadian regimen include, but are not limited to, body temperature, cortisol levels and melatonin secretion.

Thus, the pharmaceutical compositions presented herein are designed such that a window efficacy of RTK inhibitor and a window efficacy of the GC or analog of same substantially overlap.

As used in the context of this and other aspects of the present invention, the phrase “substantially overlap” with respect to the efficacy windows of the active agents means that during a certain time period upon administration of the composition described herein, both the GC or analog and the RTK inhibitor exhibit a desired pharmacological effect to some extent, namely, a plasma concentration of each agent is equal to or is higher than a minimum pharmacologically effective concentration of the agent. The efficacy windows of the active agents can overlap for at least, for example, 20 minutes, 25 minutes, 30 minutes, 1 hour, 3 hours, 5 hours, 8 hours, 10 hours, 12 hours, 15 hours, 20 hours, 24 hours, 36 hours, 48 hours, 72 hours, and even for longer time periods. According to a specific embodiment, the efficacy windows of the active agents overlap for at least 12 hours. The efficacy windows of the active agents (i.e., GC and RTK inhibitor) can overlap such that during the overlapping period, both agents exhibit a maximal efficacy, such that one agent exhibits a maximal efficacy while the other agent exhibits a partial efficacy or such that both agents exhibit a partial efficacy.

According to an embodiment of the invention, the efficacy windows of the active agents overlap for at least 10, 12 or 24 hours, so as to allow maximal activity.

As used herein, the phrase “maximal efficacy window” describes that time frame upon administration of the active agent during which the agent exhibits a maximal efficacy.

A maximal efficacy is typically related to the plasma peak concentration of an active agent.

Thus, further preferably, the composition of the present invention is designed such that a plasma peak concentration of each of the active ingredients occurs substantially simultaneously, namely, within the same time period upon administration.

One approach for achieving the above is to achieve high plasma levels of GCs. In order to achieve such staggered release, both the RTK inhibiting agent and the GC may be in a delayed release form of varying release profile, or the RTK inhibiting agent may be in immediate release form and GC in a delayed release form. According to a specific embodiment, the contemplated regimen is a day administration of the RTK inhibitors, with a delayed schedule for the GC. More specifically GCs are administered with a night schedule. For each of the active agents a specific timing of administration is optimized according to the half-life and the clearance time of the RTK inhibitors used.

In particular embodiments of the foregoing method, one or both of the administered agents (i.e., RTK inhibitor and/or GC) are approved by a national pharmaceutical regulatory agency, such as the United States Food and Drug Administration (USFDA), for administration to a human. Desirably, the compounds are administered within 12 hours of each other, within one hour of each other, or simultaneously.

According to a specific embodiment, the RTK inhibitor and/or GC are administered in the same pharmaceutical composition.

Thus, according to an aspect of the invention, there is provided a composition-of-matter comprising a therapeutically effective amount of an RTK-specific cancer therapy and a therapeutically effective amount of a glucocorticoid or glucocorticoid analog, the composition being such that an efficacy window of the RTK-specific cancer therapy and the efficacy window of the glucocorticoid or glucocorticoid analog substantially overlap.

According to a specific embodiment, the RTK-specific cancer therapy is conjugated to the glucocorticoid or glucocorticoid analog.

The RTK-specific cancer and GC can be attached to each other, directly or via a spacer, or can be otherwise associated, e.g., via, covalent bonds, electrostatic interactions, hydrogen bonding, van der Waals interactions, donor-acceptor interactions, aromatic (e.g., π-π interactions, cation-π interactions and metal-ligand interactions. These interactions lead to the chemical association of the RTK-specific cancer and GC.

As an example, GC can be attached to a protein-based RTK inhibitor (e.g., antibody) via chemical interactions with the side chains, N-terminus or C-terminus of the inhibitor.

Alternatively, the GC can be attached to the RTK inhibitor by physical association such as magnetic interactions, surface adsorption, encapsulation, entrapment, entanglement and the likes.

Alternatively, it may be desired to administer each compound individually, either by the same or different route of administration.

Thus, according to a specific embodiment, the RTK-specific cancer therapy and the glucocorticoid or glucocorticoid analog are in separate formulations.

For example, each compound may, independently, be administered by intravenous, intramuscular, subcutaneous, rectal, oral, topical, intravaginal, ophthalmic, and inhalation administration.

According to a specific embodiment, the RTK-specific cancer therapy is administered paraenterally.

According to a specific embodiment, the GC is administered orally.

Other routes of administration are provided hereinbelow.

According to a specific embodiment, each of the RTK-specific cancer therapy and the glucocorticoid or glucocorticoid analog is administered at a dose and regimen effective in treating cancer. To clarify, the GC is active in attenuating RTK signalling and not merely in ameliorating symptoms of the cancer or its treatment (e.g., immunosuppression or nausea treatment).

Thus the RTK-specific cancer therapy and the glucocorticoid or glucocorticoid analog of some embodiments of the invention can be administered to an organism per se, or in a pharmaceutical composition where it is mixed with suitable carriers or excipients.

As used herein a “pharmaceutical composition” refers to a preparation of one or more of the active ingredients (RTK-specific cancer therapy and the glucocorticoid or glucocorticoid analog) described herein with other chemical components such as physiologically suitable carriers and excipients. The purpose of a pharmaceutical composition is to facilitate administration of a compound to an organism.

Herein the term “active ingredient” refers to the RTK-specific cancer therapy and the glucocorticoid or glucocorticoid analog accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical composition which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (RTK-specific cancer therapy and the glucocorticoid or glucocorticoid analog) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient an efficacy window of RTK-specific cancer therapy and the glucocorticoid or glucocorticoid analog so as to substantially overlap. The MEC will vary for each preparation, but can be estimated from in vitro data. Dosages necessary to achieve the MEC will depend on individual characteristics and route of administration. Detection assays can be used to determine plasma concentrations.

According to a specific embodiment, administering results in an improvement in survival relative to a subject treated with the RTK-specific cancer therapy only.

According to a specific embodiment, administering results in an improvement in progression free survival relative to a subject treated with the RTK-specific cancer therapy only.

According to a specific embodiment, administering results in an improvement in overall survival relative to a subject treated with the RTK-specific cancer therapy only.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It is expected that during the life of a patent maturing from this application many relevant glucocorticoid or analogs or RTK specific cancer therapies will be developed and the scope of the terms provided herein is intended to include all such new technologies a priori.

As used herein the term “about” or “substantially” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Cell Culture and Reagents

MCF10A cells were grown as described¹⁶ and stimulated with EGF (10 ng/ml) or DEX (100 nM). siRNA transfections employed Oligofectamine (Invitrogen) and ON-Target SMART (Dharmacon, Lafayette, Colo.) oligonucleotides. Lapatinib (di-p-Toluenesulfonate Salt) was purchased from LC Laboratories. Anti GR antibody for immunostaining (sc-1004, SANTA CRUZ BIOTECHNOLOGY, INC.)

RNA Isolation, PCR and Microarrays

RNA was isolated using the PerfectPure kit from 5 Prime (Hamburg, Germany). Affymetrix GeneChip Human Exon 1.0 ST arrays were used and data were deposited in Gene Expression Omnibus (GSE53405). PCR of pre-mRNA or mRNA used forward primers positioned in the second intron or exon, respectively (Table 3 below).

TABLE 3

| | SEQ | |
|---|---|---|
| | ID | |
| Gene | NO: | Sequence 5′→3′ |
| AMIGO2_mat | 1 | ATACTGCAGCAGGGCAGAAC |
| AMIGO2_pre | 2 | TTTCTGCTTTTTACTCCCTCTGAAT |
| AMIGO2_universal | 3 | GAGTCAGATTTCCCCCTCGT |
| BHLHE40_mat | 4 | AGACGTGACCGGATTAACGA |
| BHLHE40_Pre | 5 | CCCAAAGGTGGGACTTCTCT |
| BHLHE40_universal | 6 | CAAGAACCACTGCTTTTTCCA |
| CXCL14_mat | 7 | CGCACTGCGAGGAGAAGAT |
| CXCL14_pre | 8 | ACCTCATCCTGCTCCGTTTC |
| CXCL14_universal | 9 | TTCCAGGCGTTGTACCACTT |
| CXCL1_mat | 10 | ATCCTGCATCCCCCATAGTT |
| CXCL1_pre | 11 | GAGCAGGGCAGGAGAAGAGT |
| CXCL1_universal | 12 | CTTCAGGAACAGCCACCAGT |
| DUSP1_mat | 13 | ACTTCATAGACTCCATCAAGAA |
| DUSP1_pre | 14 | GAAGGGTGTTTGTCCACTGC |
| DUSP1_universal | 15 | CTCGTCCAGCTTGACTCGAT |
| DUSP4_mat | 16 | CCACAGAGCCCTTGGACCT |
| DUSP4_pre | 17 | CCTGTGCCAAGCACTTTACC |
| DUSP4_universal | 18 | GAGGAAGGGAAGGATCTCCA |
| ENC1_mat | 19 | TTTGTCAGCACCTGGAAACA |
| ENC1_pre | 20 | CATCACACAAATCCTTCATGCT |
| ENC1_universal | 21 | AGACTTGGCCTCTCCGAAGT |
| EOMES_mat | 22 | CGCCACCAAACTGAGATGAT |
| EOMES_pre | 23 | GCCTGTTCTAGGACATCCCAATTA |
| EOMES_universal | 24 | TTGTAGTGGGCAGTGGGATT |
| EREG_mat | 25 | TCCATCTTCTACAGGCAGTCCT |
| EREG_pre | 26 | CTTCCATGAAGGCTGCAGAA |
| EREG_universal_R | 27 | AGCCACACGTGGATTGTCTT |
| ERRIF1(5′ side) | 28 | TCCTAATGGAGGTATTTCTGAATTGT |
| P_FW | | |
| ERRIF1(5′ side) | 29 | CTGGGACATCTCCAAACCTG |
| P_REV | | |
| ERRIF1(5′ side) | 30 | CCTCTTCATGTGGTCCCAAG |
| mat rev | | |
| ERRIF1(5′ side) | 31 | GCCACTGCTTTGCAGAAAAT |
| mat FW | | |
| FGD4_mat | 32 | AGCTGCTCGGAACACTTCAG |
| FGD4_pre | 33 | ACCTGATCAGTTTCCCCTATTTCT |
| FGD4_universal | 34 | TGGGCACACAGTACAGCAAC |
| FLI1_mat | 35 | TCCCTCCTCATGTCATCTCC |
| FLI1_pre | 36 | CACGGAAGTGCTGTTGTCAC |
| FLI1_Uni | 37 | TCGGTGTGGGAGGTTGTATT |
| FOXO3_mat | 38 | CTTCAAGGATAAGGGCGACA |
| FOXO3_pre | 39 | CTCGGTTTTGGACCATTCTG |
| FOXO3_universal | 40 | TCTTGCCAGTTCCCTCATTC |
| GDF15_mat | 41 | GAGCTGGGAAGATTCGAACA |
| GDF15_pre | 42 | GTTCCTGGAAAACGGTAGGC |
| GDF15_universal | 43 | CGAGAGATACGCAGGTGCAG |
| GFPT2_mat | 44 | CCTGTGCCAAGTGTGTGAGA |
| GFPT2_pre | 45 | CGGCTGGAGTACAGAGGCTA |
| GFPT2_universal | 46 | GACTTCGTGATTATTCCCATCG |
| HBEGF_mat | 47 | GCTGTGGTGCTGTCATCTGT |
| HBEGF_pre | 48 | CTTTGGAAGGACCTGCTCTG |
| HBEGF_universal | 49 | TCATGCCCAACTTCACTTTCT |
| IL8_mat | 50 | CGGAAGGAACCATCTCACTG |
| IL8_pre | 51 | AAAGGAAGTAGCTGGCAGAGC |
| IL8_universal | 52 | AGCACTCCTTGGCAAAACTG |
| HES1_mat | 53 | AAGGCGGACATTCTGGAAAT |
| HES1_pre | 54 | TGACCCGTCTGTCTCTTTCTG |
| HES1_universal | 55 | TACTTCCCCAGCACACTTGG |
| IER3_mat_F | 56 | GGACTACGCTCTGGACCTCA |
| IER3_mat_R | 57 | AGTGCGGGGAGTCACAGTTA |
| IER3_pre_F | 58 | CGACCTGACCTGTCTCCTGT |
| IER3_pre_R | 59 | GCAGAAAGAGAAGCCTTTTGG |
| IL6_mat | 60 | GCCAGAGCTGTGCAGATGAG |
| IL6_pre | 61 | CATCATCCCATAGCCCAGAG |
| IL6_universal | 62 | TCAGGGGTGGTTATTGCATC |
| IL1R1_mat_fw | 63 | TCATAGCTCTACTGATTTCTTCTCTGG |
| IL1R1_mat_rev | 64 | CGAACATCAATTTCATTTGCAG |
| IL1R1_pre_fw | 65 | ATTGCTTCCACCCTTCTTCC |
| IL1R1_pre_rev | 66 | AGGACAGGGACGAACATCAA |
| LOX_mat | 67 | CGCTGTGACATTCGCTACAC |
| LOX_pre | 68 | AAAGGTTGACTTTAAATTTGTCTGTTG |
| LOX_universal | 69 | CCATTGGGAGTTTTGCTTTG |
| MAOA_mat | 70 | TCTGACCAATTTTTCTCTTTTTGC |
| MAOA_pre | 71 | GGACAGGGTTGGAGGAAGAA |
| MAOA_universal | 72 | TGCCCAGCTCCTTAGACAAG |
| NEXN_mat | 73 | CCGAAAGAAGCAAGCTGAAG |
| NEXN_pre | 74 | TGGCTAATTCTGTGCCTTTTG |
| NEXN_universal | 75 | TGCTGTGTCTTGGTTTTCCTC |
| NRG1_mat | 76 | TGGTTCAAGAATGGGAATGAA |
| NRG1_pre | 77 | TGACACCACTTTGGTCCTGA |
| NRG1_universal | 78 | CTCTCCAGAATCAGCCAGTGA |
| PIK3R1_mat | 79 | TGTTGCACCAGGTTCTTCG |
| PIK3R1_pre | 80 | GGTGGGATTTTGTTGTTTGC |
| PIK3R1_universal | 81 | GGCAAACTGCTCTGCAAGAT |
| SCNN1B_mat | 82 | CTCCGTAGGCTTCAAGACCAT |
| SCNN1B_pre | 83 | CATTCCTTCCCCCTAACCAG |
| SCNN1B_universal | 84 | TCTCCAGGACAGCTTCCATC |
| SEMA6A_mat | 85 | AACACTGGCAATGTCAAGCA |
| SEMA6A_pre | 86 | TCAACACAGCTAGGGCATGA |
| SEMA6A_universal | 87 | TTGTCCTGGCAACGTTTTCT |
| SERPINB2_pre | 88 | TTTGATGGCTACTCAGAAGATTCA |
| SERPINB2_mat | 89 | TGGGTCAAGACTCAAACCAAA |
| SERPINB2_universal | 90 | TGGTATCCCCATCTACAGAACC |
| SLC2A14_mat | 91 | CAATGAACTTGTGGCCTGTG |
| SLC2A14_pre | 92 | TCAACCAGCTGGGCATAGTT |
| SLC2A14_universal | 93 | AGACCCAAGGATGAGTTCCAG |
| SPRY4_mat | 94 | GGCGTCTGCGAGTACAGC |
| SPRY4_pre | 95 | GGATTAGGCATCCTGCTCAA |
| SPRY4_universal | 96 | CTGAGCATCAGGCTGCAAAC |
| VEGFA_mat | 97 | AGGAGGAGGGCAGAATCATC |
| VEGFA_pre | 98 | GCATTACAGAGCTGGGTGGA |
| VEGFA_universal | 99 | AGCTGCGCTGATAGACATCC |
| TNFAIP3_mat | 100 | ACCCTGGAAAGCCAGAAGAA |
| TNFAIP3_pre | 101 | TGCTGGGTCTTACATGCAGAT |
| TNFAIP3_universal | 102 | CTGAACGCCCCACATGTACT |
| TBP_F | 103 | CTTCACACGCCAAGAAACAGT |
| TBP_R | 104 | GCTGGCCCATAGTGATCTTT |
| TGFA_2_pre_F | 105 | CCCTGGAGAGCTAGGGTAACA |
| TGFA_2_mat_F | 106 | GTTTTTGGTGCAGGAGGACA |
| TGFA_2_universal_R | 107 | CACCAACGTACCCAGAATGG |

All reactions were performed using Power SYBR Green PCR Master Mix (Applied Biosystems, Foster City, CA). For high throughput PCR the Fluidigm® BioMark system was employed. The Affymetrix Expression Console was used for analyses of DNA-arrays, as described⁵¹.

Cell Migration Assays

Cells (5×10⁴ cell/insert) were plated in the upper compartment of a 24-well transwell tray (Corning, Acton, MA), and their migration was assayed¹⁶. Cell invasion assays were performed using BioCoat Matrigel Invasion Chambers (BD Bioscience, Franklin Lakes, N.J.). For tracking, cells were seeded (3×10³ cells/cm²) on collagen-coated micro-slide (from Ibidi). For collective migration, 8×10⁴ cells were seeded in plastic insets (Ibidi), and after overnight incubation, the plastic barriers were removed and time-lapse images were recorded.

Analyses of Human Specimens

Immunohistochemistry of formalin-fixed, paraffin-embedded tissues was performed using the Envision Detection System (DakoCytomation, Carpinteria, Calif.). Following antigen retrieval, an anti-ERK or an anti-GR antibody (NCL-GCR, Novocastra) was added and incubated overnight at 4° C. After immunostaining, slides were counterstained with Mayer's haematoxylin (Sigma-aldrich). Two pathologists independently assessed protein levels. Statistical analysis of the data was done using the SPSS suite. Patient survival analysis was performed on a previously described cohort (Curtis et al., 2012). The Chi-square test was used for association analysis between categorical variables, and a Cox model was fitted to the data using breast cancer specific death as an endpoint.

Animal Studies

All animal experiments were approved by the institutional committee. C57BL/6 (CD45.2, Harlan) and knockout animals were maintained under defined flora conditions and at 12 hour light-dark cycles. For daily clock studies, female mice (10-12 week old) were divided into 2 groups; one was maintained in the day-night room, and the second group was located in a special room (with inverted day-light cycles). Mice were let acclimate for at least one week prior to protein and RNA extraction. For tumor xenograft studies, 20 athymic nude (nu/nu) mice were used and maintained in a Specific Pathogen Free environment. Animals (n=10 per group) were inoculated subcutaneously in the left leg (using a sterile 22-gauge needle) with 5×10⁶ N87 cells. Mice were randomized into two groups, daily treated with Lapatinib by oral gavage (40 mg/kg) in the night (2 hours after the light off) and in the day (approximately 70 minutes before light on). Treatments were started 2 weeks after cell injection. Tumor width (W) and length (L) were measured once a week with a calliper and tumor volume (V) was calculated according to the formula: V=0.5×W²×L.

Nuclear-Cytoplasm Fractionation

Cells were harvested in a hypotonic buffer (10 mM HEPES pH 7.9, 1.5 MgCl₂, 10 mM KCl, 0.5 mM DTT, 0.5% NP40 and 1 μM sodium vanadate) containing a mixture of protease inhibitors. Nuclei were centrifuged and resuspended in lysis buffer, followed by a sonication step.

### Example 2

### Ligand-Activated GRs Inhibit EGF-Induced Motility of Mammary Cells

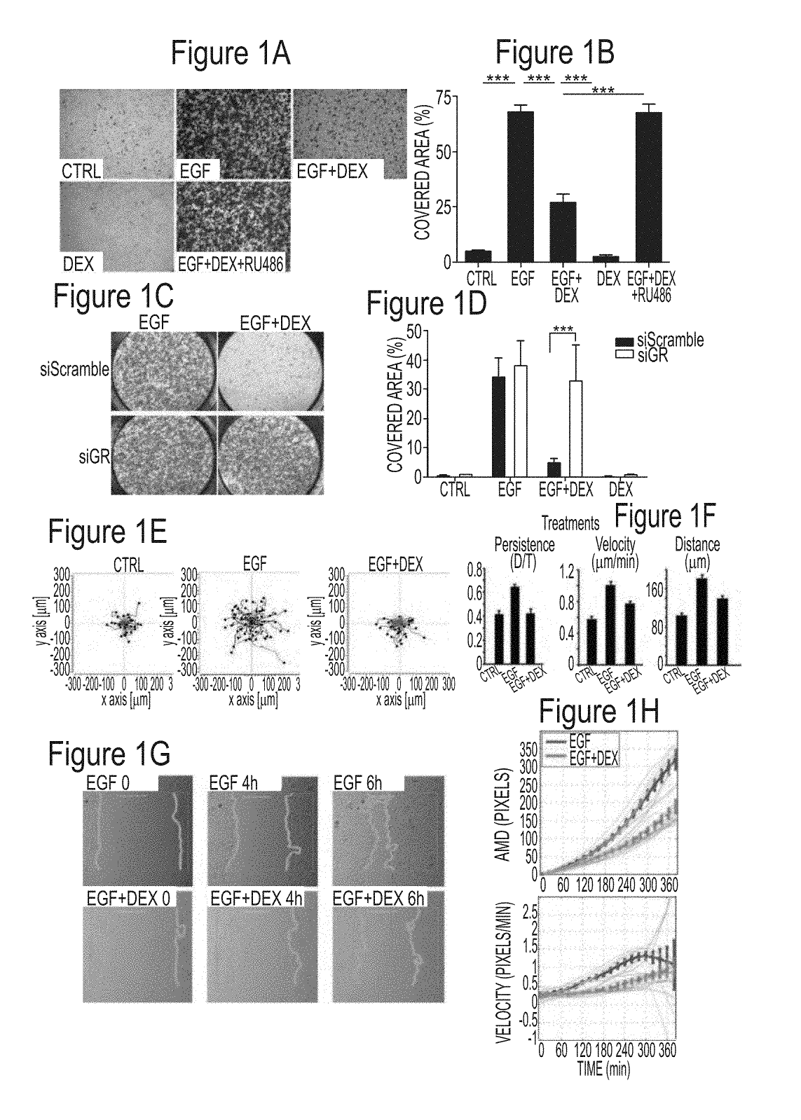

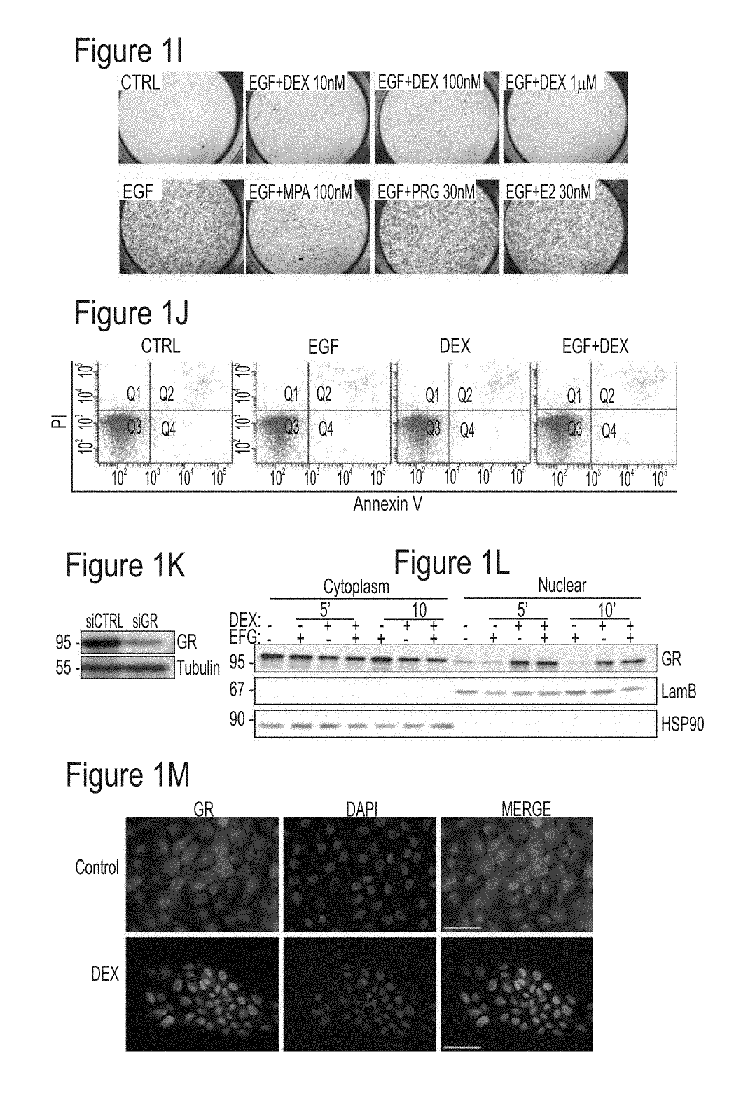

On stimulation with EGF, MCF10A ATCC CRL-10317 mammary epithelial cells initiate transcriptional programs culminating in migration and invasion^{15,16}. To examine potential interactions between the EGFR pathway and steroid hormone signalling, MCF10A cells were plated in transwell trays and treated with EGF, in the presence of estradiol (E2), progesterone (PRG), medroxyprogesterone acetate (MPA), a synthetic variant of progesterone, or dexamethasone (DEX), a synthetic GC (FIG. 1E). The results identified DEX as a potent inhibitor of EGF-induced cell migration. MPA was less potent and both E2 and PRG displayed weak or no activity, probably due to the absence of the respective receptors. Importantly, markers of apoptosis (annexin V) and necrosis (propidium iodide) excluded the possibility of cellular toxicity (FIG. 1J).

While DEX is specific for GR, MPA binds the progesterone, androgen and glucocorticoid receptors with EC50 values of approximately 0.01, 1, and 10 nM, respectively. Hence, migration inhibition by DEX and, to some extent, by MPA could be mediated by GR, as supported by using RU486, a GR antagonist (FIGS. 1A and 1B). In addition, GR-specific siRNAs that reduced receptor expression by approximately 80% (FIG. 1K), inhibited the effect of DEX on migration (FIGS. 1C and 1D). Hence, it is suggested that ligand-bound GRs inhibit EGF-induced cell migration by translocating to the nucleus and modulating transcriptional events. In line with this model, both subcellular fractionation and immunofluorescence confirmed rapid (within 5 minutes) translocation of GRs to the nuclei of DEX-stimulated cells, independent of EGF (FIGS. 1L-M).

Next, the question whether DEX treatment alters migration directionality, namely the ability of cells to maintain a migration course¹⁷ was assessed. Quantification of directionality relates the linear distance between the start and end points (D) to the total distance (T) travelled. The rose plots of cellular tracks (FIGS. 1E and 1F) indicated that EGF enhanced directional persistence (D/T) and accelerated velocity, but DEX abolished these effects. To complement these observations, a wound closure assay was performed, as recently described¹⁸ (FIGS. 1G and 1H). The results confirmed inhibition of collective cell migration, and showed a delay of migration onset by approximately 2 hours, suggesting that DEX induces migration-inhibitory transcriptional programs.

### Example 2

### Transcriptional Programs Stimulated By GR and EGFR Exhibit Modular Structures and Display a Complex Mode of Mutual Interference

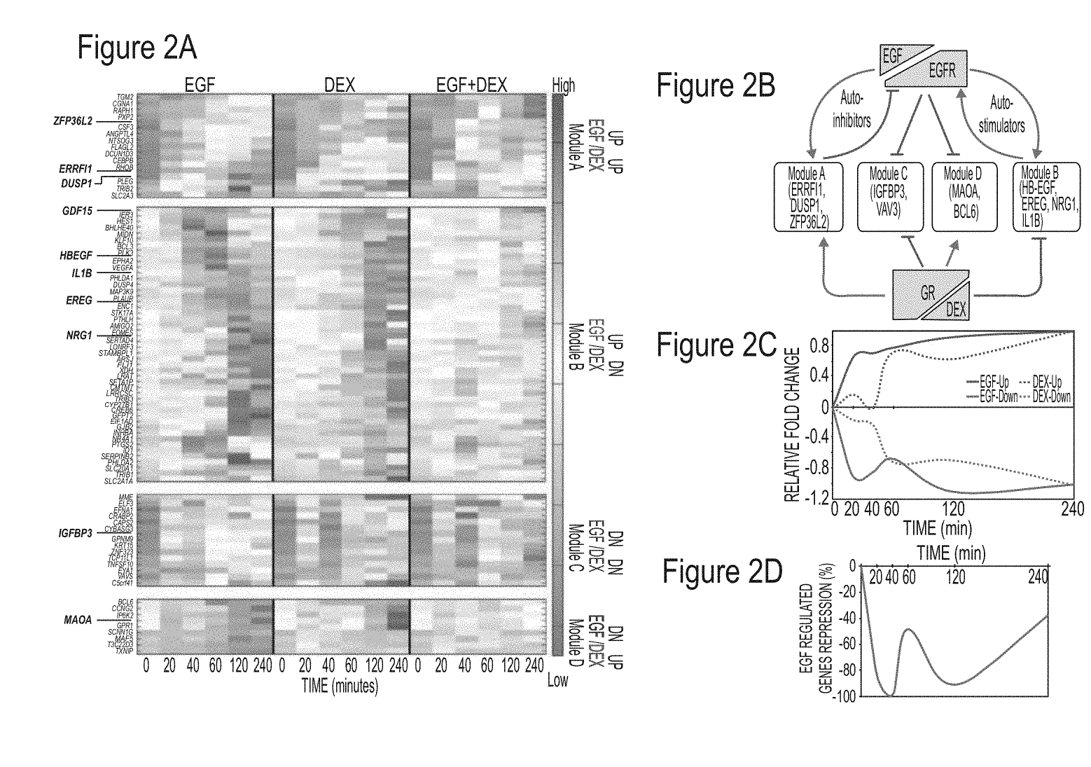

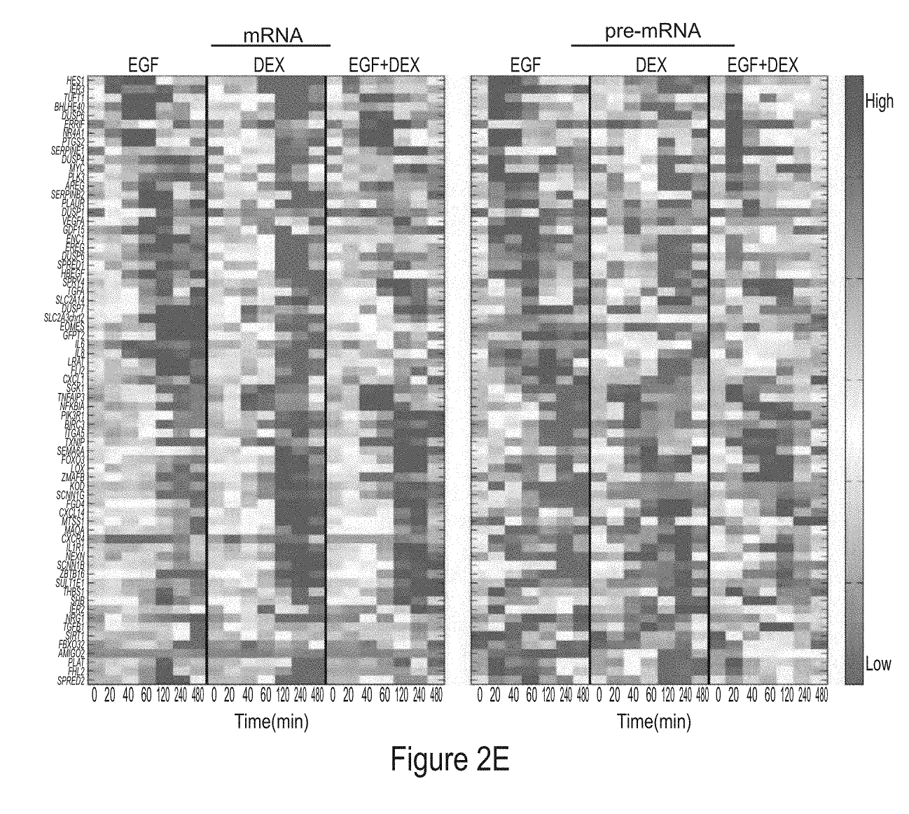

Conceivably, the inhibitory effect of GR involves alterations of EGF-induced transcription. Specifically, GR might affect transcript synthesis or modulate EGF-induced RNA splicing in MCF10A cells. To address such models, cells were stimulated with EGF, DEX or the combination, mRNAs were isolated along a time course from 20 minutes to four hours, and the RNA was hybridized to Affymetrix Exon Arrays which is able to resolve small changes in splicing¹⁹. The results obtained are summarized in FIG. 2A. In addition, confirmatory PCR analyses are provided. Notably, the combined treatment exerted no marked effects on RNA splicing. To cluster other transcriptional events, a set of logical rules was applied to define modules of active genes (FIG. 2B):
Module A: Transcripts up-regulated by both EGF and DEX (EGF^{UP}/DEX^{UP})
Module B: Transcripts up-regulated by EGF but downregulated by DEX (EGF^{UP}/DEX^{DN})
Module C: Transcripts downregulated by both agents (EGF^{DN}/DEX^{DN})
Module D: Transcripts downregulated by EGF and up-regulated by DEX (EGF^{DN}/DEX^{UP})

Interestingly, it is noted that Module A (EGF^{UP}/DEX^{UP}) included several inducible inhibitors of EGFR, such as ERRFI1/MIG6, ZFP36L2 and DUSP1, which are normally engaged in delayed feedback inhibition of EGFR signalling⁵. Conceivably, their induction by GR represents an effective inhibitory strategy. Consistent with this logic, Module B (EGF^{UP}/DEX^{DN}) includes positive feedback regulators of the EGFR pathway, such as neuregulin 1 (NRG1), HB-EGF and EREG which sustain EGFR signalling²⁰. In conclusion, GR orchestrates a transcriptional response resulting in downregulation of several positive EGFR regulators (Module B) coupled with up-regulation of multiple EGFR inhibitors (Module A), thereby robustly terminates EGFR signalling.

Comparison of the temporal patterns of EGF-and DEX-regulated genes indicated that the onset of EGF-induced, or repressed, transcripts was very fast in comparison to the effect of DEX. The latter displayed a 40-min long delay (FIG. 2C). In addition, the inhibitory effect of DEX on EGF-induced genes reached 70% of maximal capacity already at 20 minutes (FIG. 2D), significantly earlier than the peak of changes induced by DEX alone. Altogether, these observations raised the possibility that GR intercepts, likely by means of transrepression, specific TFs that undergo post-translational modifications downstream to EGFR signalling.

### Example 3

### GR Exploits a Feedback Module that Normally Terminates RTK Signalling

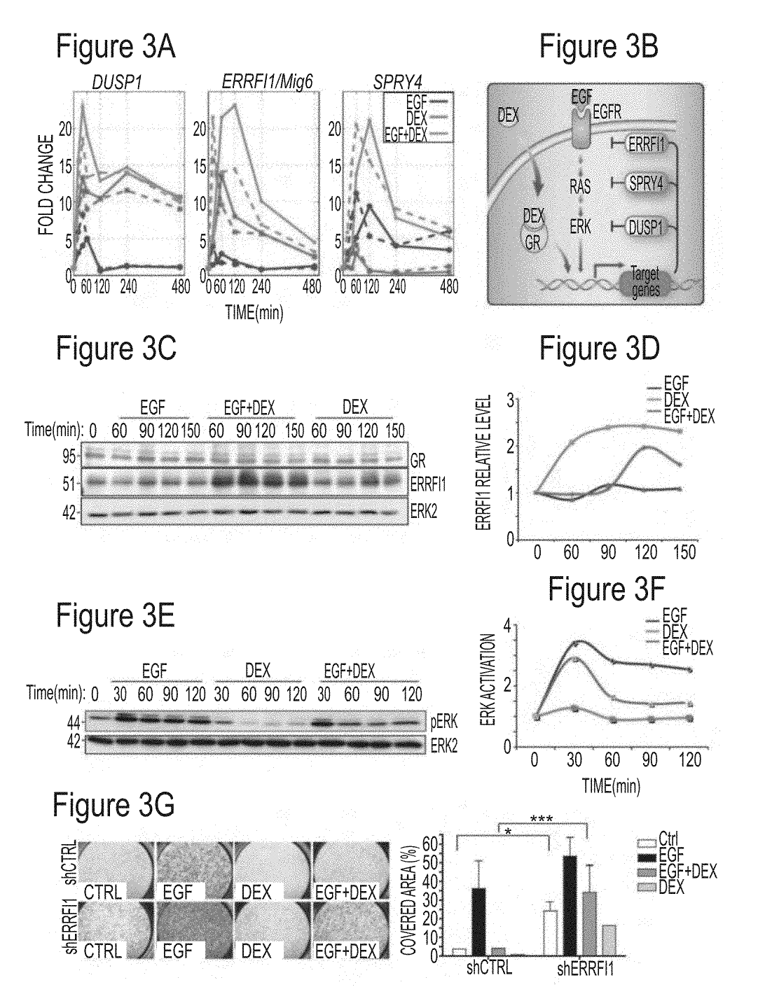

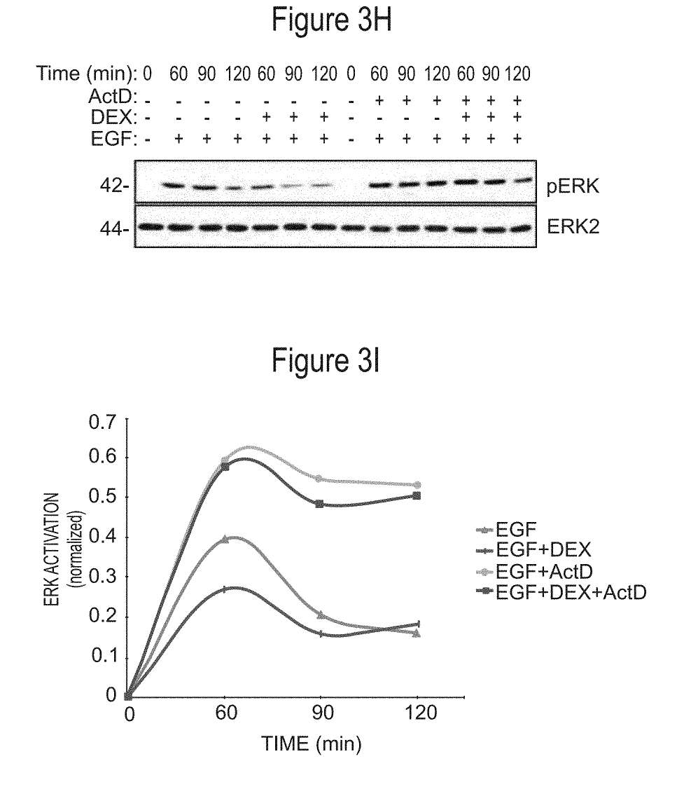

Analyses of defects in vulva formation in worms and aberrations in eye development in insects, two processes controlled by EGFR, helped define several evolutionary conserved and partly redundant negative feedback loops able to robustly terminate EGFR signalling²¹. Since Module A (EGF^{UP}/DEX^{UP}) includes several orthologs of the invertebrate negative feedback loops, three of them were selected for further analysis. DUSP1 is the prototype of MAPK-specific phosphatases, which dephosphorylate the shared Thr-Xxx-Tyr motif of MAPKs. ERRFI1/MIG6 (also called RALT) is a previously identified steroid-inducible adaptor, which physically binds and inhibits the kinase domain of EGFR⁵. The third feedback regulator studied was sprouty 4 (SPRY4), a member of the small family of adaptors able to specifically inhibit RAS-to-ERK signalling²². By using primers specific for the nascent or the mature transcripts, de novo transcription of these three negative regulators was followed (FIG. 3A). The precursor and mature transcripts exhibited similar profiles, but unlike the relatively transient and weak induction of DUSP1 and ERRFI1 by EGF (3-5 fold), treatment of cells with DEX, and especially with the DEX+EGF combination, strongly enhanced and prolonged the up-regulation signal (20-25 fold). Because each feedback regulator acts at a different level of the signalling cascade (FIG. 3B), and all three were rapidly induced, the enhanced and prolonged induction by the DEX+EGF combination likely translates to robust inhibition of the RTK-to-ERK signalling pathway. This possibility was further examined by focusing on ERRFI1.

Consistent with the gene expression data, immunoblotting confirmed strong up-regulation of the ERRFI1 protein in cells co-treated with EGF and DEX (FIG. 3C). Similarly, quantification of the signals indicated that the combined treatment induced an earlier and more sustained activation of ERRFI1 (FIG. 3D). Because the three regulators selected for analysis act upstream to ERK, the status of active ERK (pERK) was examined (FIGS. 3E and 3F). EGF rapidly stimulated ERK, but the addition of DEX reduced the amplitude and markedly shortened the duration of ERK activation. This effect appeared to depend on de novo transcription, since DEX was unable to reduce ERK activation in the presence of a transcription inhibitor, actinomycin D (FIG. 3H-I).

To monitor functional consequences of the GR-to-RTK crosstalk, ERRFI1 expression was stably reduced; and the ability of DEX to inhibit EGF-induced migration was tested (FIG. 3G). Interestingly, under basal (unstimulated) conditions ERRFI1-depleted cells displayed higher migration relative to the control cells. Nevertheless, EGF still increased migration of ERRFI1-depleted cells, but the inhibitory effect of DEX was much smaller compared to control cells. Whereas DEX inhibited migration of control cells by 90%, this effect was diminished to 30% in ERRFI1-depleted cells. In conclusion, GR activation involves up-regulation of a well-characterized group of negative feedback regulators of EGFR signalling. In line with the critical roles played by EGFR's feedback regulators in GR signalling, intervening with the function of just one of these regulators, ERRFI1, significantly limited the ability of GR to inhibit EGFR signalling.

### Example 4

### GR Employs Repression Mechanisms to Regulate Transcription of Module B Genes

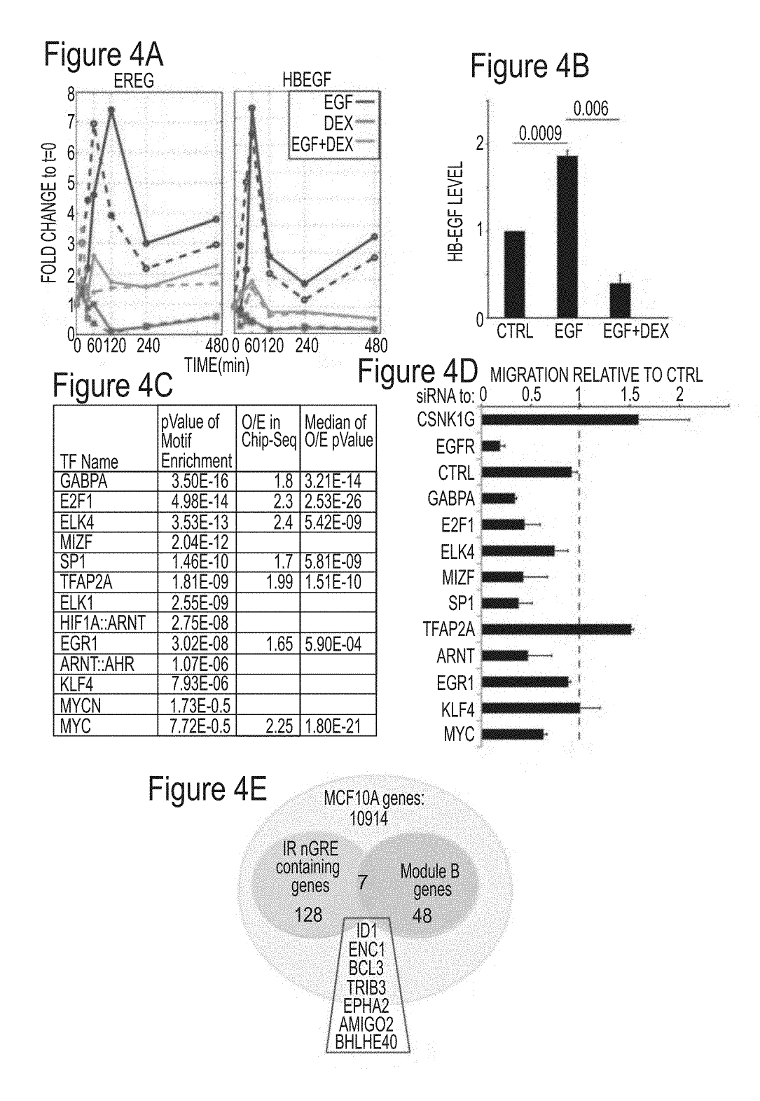

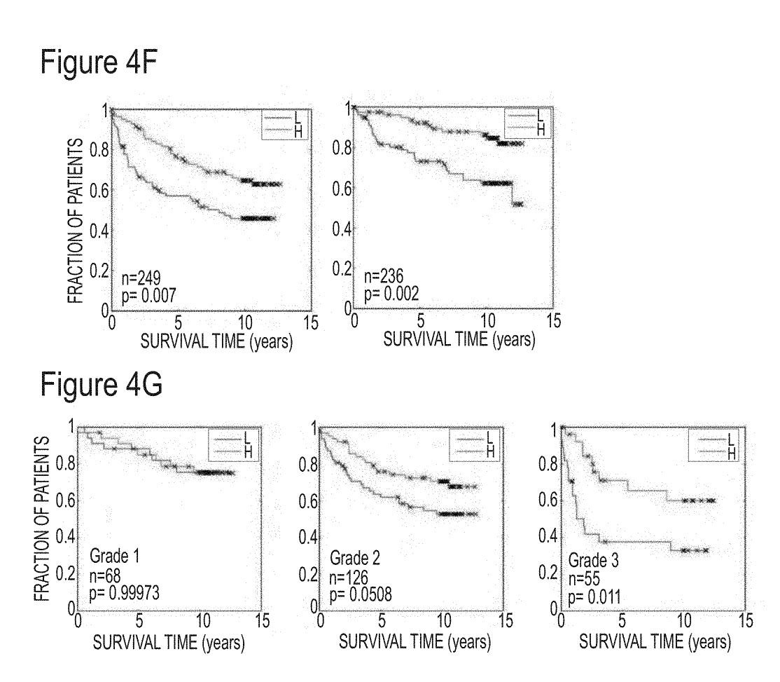

EGF-dependent transcriptional responses are characterized by early induction of auto-stimulatory loops comprising several growth factors, such as TGFA, NRG1, EREG and HBEGF, which not only auto-stimulate EGFR, but also engage additional EGFR family members²⁰. DEX strongly inhibited these auto-stimulatory loops, as detected by real time and immunological assays (FIGS. 4A and 4B). The observed rapid effects of DEX on the levels of both pre-mRNA and mRNA levels raised the possibility that GR transrepresses pre-existing immediate early transcription factors (IETFs) responsible for regulation of EGFR ligands and other module B genes. To examine this, TF binding motifs over-represented in the promoters of Module B genes were identified, and then the results were validated by using Cscan, a software based on extensive chromatin immunoprecipitation experiments (FIG. 4C). In the next step, each protein of the resulting list was functionally tested by using siRNAs and a migration assay. The results presented in FIG. 4D indicated that depletion of the majority of candidates reduced EGF-induced migration, in line with a transrepression model that repeatedly engages a relatively small group TFs to inhibit EGFR signalling. Interestingly, some of the predicted TFs, such as GABPA, ELK1 and ELK4, belong to the ETS family, while others (e.g., SP1 and E2F1) are frequently regulated by growth factors.

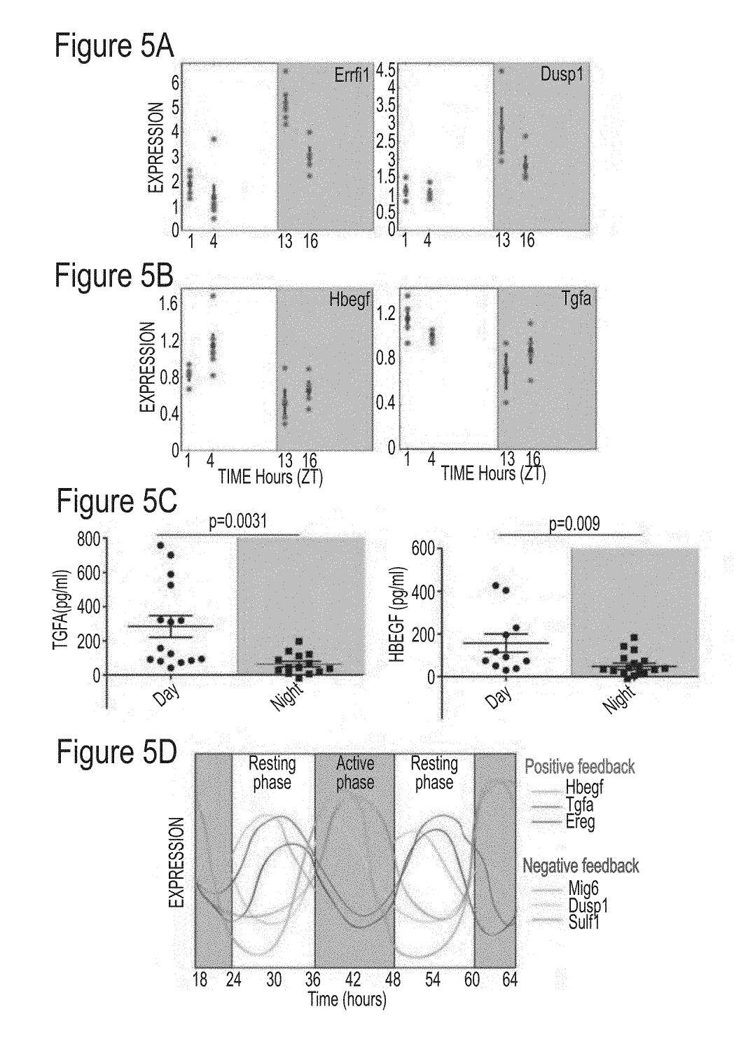

Along with physical tethering of specific TFs, like NF-κB and STAT5¹⁰, GR might induce direct repression via binding to palindromic sequences consisting of two inverted repeated motifs, IR nGREs, which are cis-acting response elements⁸. While probing MCF10A cells, 128 IR nGRE-containing genes (approximately 1% of all expressed genes) were identified. Astonishingly, by focusing only on the Module B genes, the enrichment for IR nGREs reached 15% (p=1.2781e-06; FIG. 5F). For example, this group encodes BCL3, which regulates NF-κB target genes²³. In conclusion, these findings offer two GR-mediated modes of suppressing RTK signalling: first, by transrepressing pre-existing TFs, and second by binding to IR nGREs.

### Example 5

### Daily Oscillations of Glucocorticoids Control EGFR's Transcriptional Programs In Vivo

Next, the crosstalk between GR and the RTK pathway was explored in vivo. GCs exhibit a daily rhythm, which affects behavioural patterns²⁴, and this oscillation has generally been attributed to the hypothalamus-pituitary-adrenal (HPA) neuroendocrine axis. The oscillation profile has a characteristic pattern, with a peak in the beginning of the active, dark phase in rodents. To examine the prediction that GCs control expression of EGFR's negative regulators, mRNA levels of two Module A genes, Errfil and Duspl, were analyzed in mouse livers. In support of a suppressive crosstalk, these regulators displayed daily oscillations with amplitudes of 2-4 fold change and higher levels in the active, nocturnal phase (FIG. 5A). By contrast, two EGFR positive regulators, Hbegf and Tgfa, displayed reciprocal patterns in lungs, peaking during the resting (diurnal) phase (FIG. 5B). Using ELISA and mouse blood samples collected during the diurnal (ZT4-ZT10) and nocturnal (ZT15-ZT20) phases, the possibility that the levels of HB-EGF and TGF-alpha oscillate in a circadian manner was supported (FIG. 5C). Furthermore, compilation of experimental data from expression arrays available through Circa DB, the circadian expression profiles database (bioinfdotitmatdotupenndotedukirca/query), confirmed antithetical oscillations of EGFR's negative (Mig6, Dusp1, Sulf1) and positive (Tgfa, Hbegf, Ereg) feedback regulators, as determined by analyzing a set of four different murine tissues (FIG. 5D). In summary, both positive and negative feedback regulators of RTK signalling display oscillatory patterns in vivo, in line with diurnal secretion of the activators, namely EGFR ligands, coupled to nocturnal synthesis of several intracellular inhibitors of EGFR signalling, to achieve robust suppression and activation of EGFR signalling during the active (nocturnal) and resting (diurnal) phases, respectively, in rodents.

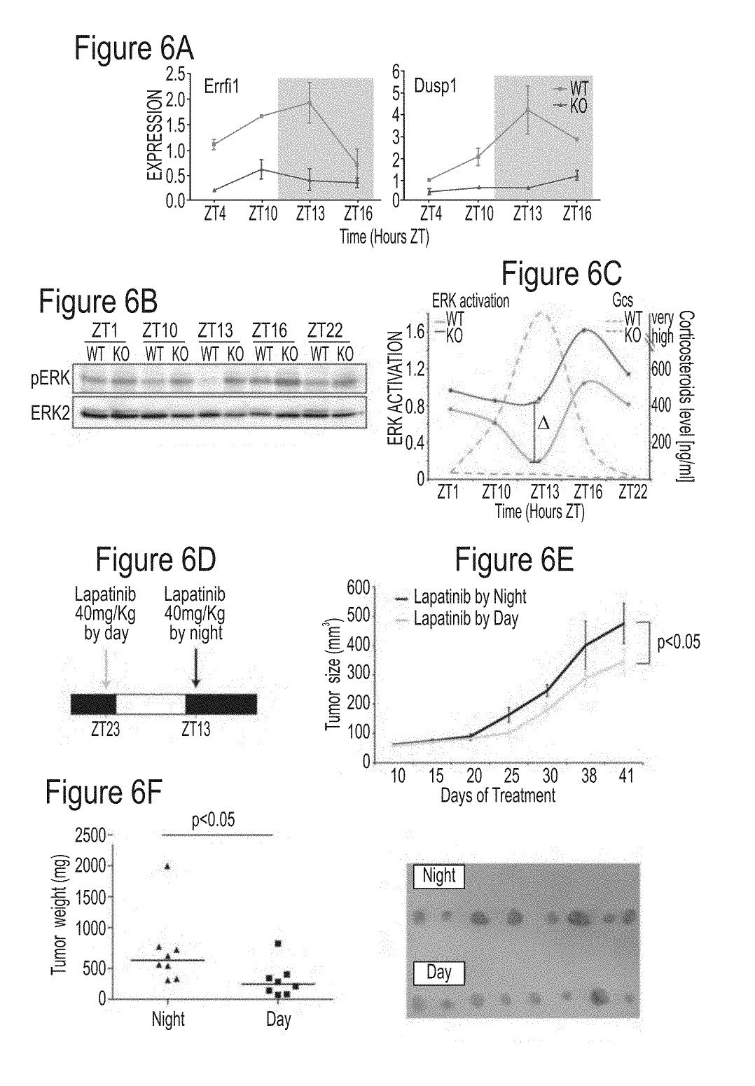

To corroborate these conclusions, a murine model with aberrant GC production was employed. CRFR1 encodes one of two receptors for the corticotropin releasing factor, which maintains the HPA axis. Homozygous CRFR1-depleted mice (Crfr1^{-/-}) display constantly low plasma corticosterone concentrations resulting from agenesis of the zona fasciculata region of the adrenal gland²⁵. Hence, this animal model represents a suitable system for addressing the GR-to-RTK crosstalk. In line with other lines of evidence, the expression levels of two negative feedback regulators, Errfil and Duspl, were generally reduced in livers isolated from in Crfr1 mutant mice and they lacked the circadian fluctuations observed in control mice (FIG. 6A). These results suggested that EGFR signalling is under control of the HPA neuroendocrine axis. Hence, in the next step the activation of ERK, a downstream effector of EGFR, was tested in liver extracts collected around the clock from wild type and mutant animals (FIGS. 6B and 6C). Interestingly, the Crfr1 mutant mice displayed normal ERK activation, but they lacked the inactivation phase (marked by A in FIG. 6C), which coincides with the peak of corticosteroid concentration in blood. Moreover, in line with the suppressive action of GR, ERK displayed overall higher levels in the mutants compared to WT animals. Altogether, the comparison between wild type and the Crfr1 mutant mice supported the possibility that negative modulators of EGFR (i.e., Errfil) and MAPK (i.e., Duspl) are controlled in vivo by ligands of GR.

### Example 6

### A Clinically Approved Anti-EGFR Drug Better Inhibits Tumor Xenogratfs if Administered at the Resting Phase

Constitutive signals generated by EGFR and its family member, called HER2 or ERBB2, drive several types of tumors, hence drugs intercepting these signals are active in patients whose tumors display aberrant forms of these RTKs^{26,27}. Lapatinib, an oral low molecular weight drug approved for breast cancer treatment, specifically inhibits the tyrosine kinase activities of both EGFR and HER2²⁸. The working hypothesis predicted that administration of Lapatinib at the beginning of the resting (diurnal) phase of mice carrying xenografts of an EGFR/HER2-driven tumor would better inhibit tumorigenic growth relative to administration during the active phase, in which EGFR signalling is anyhow robustly suppressed by liganded GRs. As a model xenograft, N87 human gastric cancer cells NCI-N87 ATCC CRL-5822, which are sensitive to HER2-targeting drugs²⁹ were selected. Mice (CD1/nude) were injected subcutaneously with N87 cells, and once tumors became palpable we randomized the animals into several groups. The “day” group received Lapatinib by oral gavage, just before the beginning of the resting phase, while the “night” group was treated at the beginning of the active phase (see a scheme in FIG. 6D). Tumor sizes were followed over a period of several weeks, and their weights were inspected in the end of the trial (FIGS. 6E-6F). The results confirmed statistically significant enhancement of Lapatinib therapeutic impact when administered just before the resting phase (ZT23), as expected by the suppressive GR-to-RTK crosstalk. Interestingly, tumors differed not only by their size but also be their appearance, suggesting that the administration of Lapatinib during the resting phase also inhibited tumor angiogenesis (FIG. 6F; right panel), in line with a similar effect of an anti-HER2 antibody when tested in animals³⁰. Taken together with the in vitro studies and observations made with genetically modified mice, the effect of timing on drug efficacy not only adds another line of evidence in support of the model, but also proposes a potential strategy capable of augmenting the therapeutic effects of anti-cancer drugs.

### Example 7

### High Abundance of GR Associates with Lower ERK Activity and Longer Survival of Breast Cancer Patients

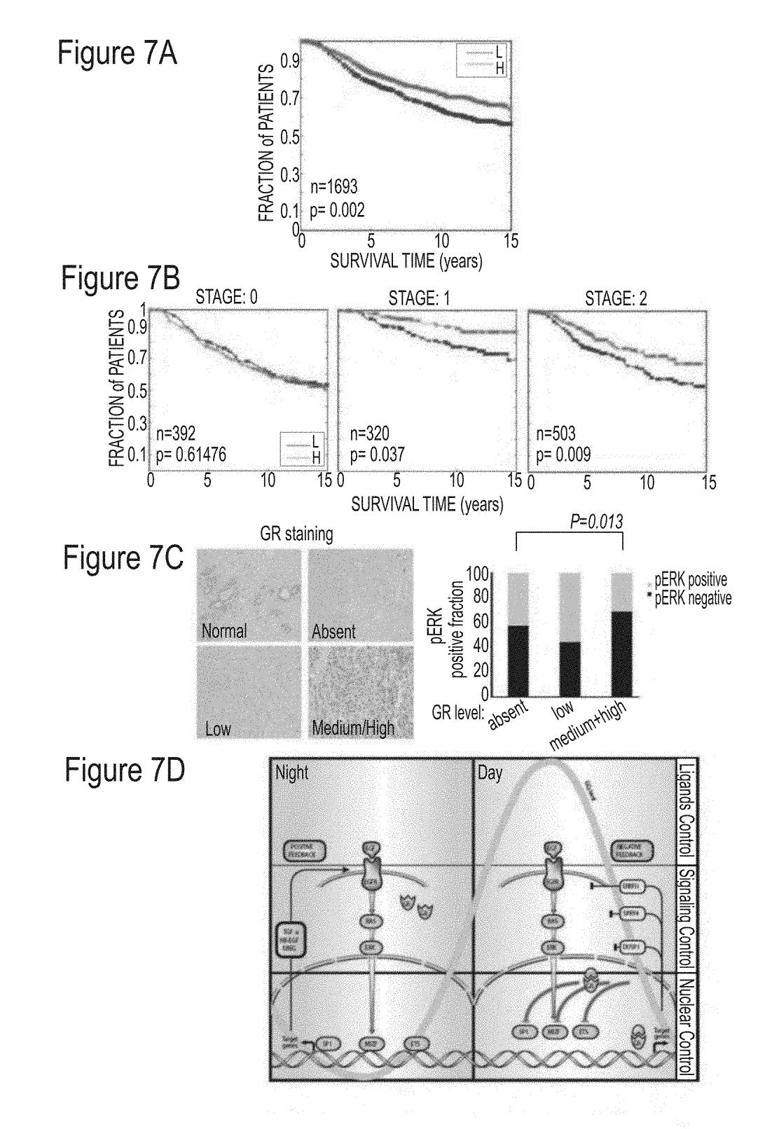

Since EGFR and other RTKs play pivotal roles in progression of human breast cancer³, and because the present results indicated that GR signalling suppresses RTKs, GR's prognostic significance was addressed in tumor specimens. Analysis of approximately 1,700 patients of the METABRIC dataset³¹ associated high abundance of GR (NR3C1) with longer patient survival time (p=0.002; FIG. 7A). These relations were confirmed in two independent, but smaller groups of patients (FIGS. 4F-G). Notably, a previous study associated longer relapse-free survival with higher GR expression in a group of 87 patients, but this was limited to ER-positive patients³². Interestingly, stratifying patients of the METABRIC cohort according to disease stage indicated that low GR expression predicts poor survival only in advanced disease stages of disease (FIG. 7B), and similar analysis of two smaller cohort of patients^{33,34} showed that low GR associates with poor prognosis only in grade 2 and grade 3 patients, but no such association was found in the grade 1 group (FIG. 4F), raising the possibility that loss of GR occurs late in breast cancer progression.

To relate these observations to the emerging notion that GR suppresses RTK signalling, 362 breast cancer specimens were immunostained for both GR and the active form of ERK. Tumors were scored, on the one hand, as phospho-ERK positive or negative, and on the other hand as high/medium GR, low GR or undetectable GR levels. This analysis clearly indicated an inverse correlation between GR abundance and ERK activation (FIG. 7C; p=0.013). In conclusion, low abundance of GR associates with both higher ERK activation and poorer prognosis, suggesting that the corresponding patients suffer from more aggressive disease because of unrestrained RTK-to-ERK signalling.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Listed Throughout the Application

1 Troyer, K. L. & Lee, D. C. Regulation of mouse mammary gland development and tumorigenesis by the ERBB signalling network. Journal of mammary gland biology and neoplasia 6, 7-21 (2001).
2 Wintermantel, T. M., Bock, D., Fleig, V., Greiner, E. F. & Schutz, G. The epithelial glucocorticoid receptor is required for the normal timing of cell proliferation during mammary lobuloalveolar development but is dispensable for milk production. Mol Endocrinol 19, 340-349 (2005).
3 Hynes, N. E. & Watson, C. J. Mammary gland growth factors: roles in normal development and in cancer. Cold Spring Harbor perspectives in biology 2, (2010).
4 Avraham, R. & Yarden, Y. Feedback regulation of EGFR signalling: decision making by early and delayed loops. Nature Review Molecular Cell Biology 12, 104-117 (2011).
5 Segatto, O., Anastasi, S. & Alema, S. Regulation of epidermal growth factor receptor signalling by inducible feedback inhibitors. Journal of cell science 124, 1785-1793 (2011).
6 Bookout, A. L. et al. Anatomical profiling of nuclear receptor expression reveals a hierarchical transcriptional network. Cell 126, 789-799, (2006).
7 Kadmiel, M. & Cidlowski, J. A. Glucocorticoid receptor signalling in health and disease. Trends in pharmacological sciences 34, 518-530 (2013).
8 Surjit, M. et al. Widespread negative response elements mediate direct repression by agonist-liganded glucocorticoid receptor. Cell 145, 224-241 (2011).
9 Voss, T. C. & Hager, G. L. Dynamic regulation of transcriptional states by chromatin and transcription factors. Nat Rev Genet 15, 69-81 (2014).
10 Stocklin, E., Wissler, M., Gouilleux, F. & Groner, B. Functional interactions between StatS and the glucocorticoid receptor. Nature 383, 726-728 (1996).
11 Herr, I., Gas sler, N., Friess, H. & Buchler, M. W. Regulation of differential pro-and anti-apoptotic signalling by glucocorticoids. Apoptosis: an international journal on programmed cell death 12, 271-291 (2007).
12 Greene, M. W. Circadian rhythms and tumor growth. Cancer Lett 318, 115-123 (2012).
13 Hermes, G. L. et al. Social isolation dysregulates endocrine and behavioral stress while increasing malignant burden of spontaneous mammary tumors. Proceedings of the National Academy of Sciences of the United States of America 106, 22393-22398 (2009).
14 Holsboer, F. & Ising, M. Stress hormone regulation: biological role and translation into therapy. Annual review of psychology 61, 81-109 (2010).
15 Muthuswamy, S. K., Li, D., Lelievre, S., Bissell, M. J. & Brugge, J. S. ErbB2, but not ErbB1, reinitiates proliferation and induces luminal repopulation in epithelial acini. Nature cell biology 3, 785-792 (2001).
16 Tarcic, G. et al. EGR1 and the ERK-ERF axis drive mammary cell migration in response to EGF. FASEB journal: official publication of the Federation of American Societies for Experimental Biology 26, 1582-1592 (2012).
17 Pegtel, D. M. et al. The Par-Tiaml complex controls persistent migration by stabilizing microtubule-dependent front-rear polarity. Current biology: CB 17, 1623-1634 (2007).
18 Zeisel, A. et al. qCMA: A Desktop Application for Quantitative Collective Cell Migration Analysis. Journal of biomolecular screening, doi:10.1177/1087057112461940 (2012).
19 Kostler, W. J. et al. Epidermal growth-factor—induced transcript isoform variation drives mammary cell migration. PloS one 8, e80566 (2013).
20 Wilson, K. J., Gilmore, J. L., Foley, J., Lemmon, M. A. & Riese, D. J., 2nd. Functional selectivity of EGF family peptide growth factors: implications for cancer. Pharmacology & therapeutics 122, 1-8 (2009).
21 Freeman, M. Feedback control of intercellular signalling in development. Nature 408, 313-319 (2000).
22 Mason, J. M., Morrison, D. J., Basson, M. A. & Licht, J. D. Sprouty proteins: multifaceted negative-feedback regulators of receptor tyrosine kinase signalling. Trends Cell Biol 16, 45-54 (2006).
23 Zhang, Q., Didonato, J. A., Karin, M. & McKeithan, T. W. BCL3 encodes a nuclear protein which can alter the subcellular location of NF-kappa B proteins. Molecular and cellular biology 14, 3915-3926 (1994).
24 Ikeda, Y., Kumagai, H., Skach, A., Sato, M. & Yanagisawa, M. Modulation of Circadian Glucocorticoid Oscillation via Adrenal Opioid-CXCR7 Signalling Alters Emotional Behavior. Cell 155, 1323-1336 (2013).
25 Smith, G. W. et al. Corticotropin releasing factor receptor 1-deficient mice display decreased anxiety, impaired stress response, and aberrant neuroendocrine development. Neuron 20, 1093-1102 (1998).
26 Arteaga, C. L. et al. Treatment of HER2-positive breast cancer: current status and future perspectives. Nature reviews. Clinical oncology 9, 16-32 (2012).
27 Yarden, Y. & Pines, G. The ERBB network: at last, cancer therapy meets systems biology. Nature reviews. Cancer 12, 553-563 (2012).
28 Spector, N. L. et al. Study of the biologic effects of lapatinib, a reversible inhibitor of ErbB1 and ErbB2 tyrosine kinases, on tumor growth and survival pathways in patients with advanced malignancies. Journal of clinical oncology : official journal of the American Society of Clinical Oncology 23, 2502-2512 (2005).
29 Ben-Kasus, T., Schechter, B., Lavi, S., Yarden, Y. & Sela, M. Persistent elimination of ErbB-2/HER2-overexpressing tumors using combinations of monoclonal antibodies: relevance of receptor endocytosis. Proceedings of the National Academy of Sciences of the United States of America 106, 3294-3299 (2009).
30 Izumi, Y., Xu, L., di Tomaso, E., Fukumura, D. & Jain, R. K. Tumour biology: herceptin acts as an anti-angiogenic cocktail. Nature 416, 279-280 (2002).
31 Curtis, C. et al. The genomic and transcriptomic architecture of 2,000 breast tumours reveals novel subgroups. Nature 486, 346-352 (2012).
32 Pan, D., Kocherginsky, M. & Conzen, S. D. Activation of the glucocorticoid receptor is associated with poor prognosis in estrogen receptor-negative breast cancer. Cancer research 71, 6360-6370 (2011).
33 Ivshina, A. V. et al. Genetic reclassification of histologic grade delineates new clinical subtypes of breast cancer. Cancer research 66, 10292-10301 (2006).
34 Miller, L. D. et al. An expression signature for p53 status in human breast cancer predicts mutation status, transcriptional effects, and patient survival. Proceedings of the National Academy of Sciences of the United States of America 102, 13550-13555 (2005).
35 Yang, Y. et al. Sequential requirement of hepatocyte growth factor and neuregulin in the morphogenesis and differentiation of the mammary gland. The Journal of cell biology 131, 215-226 (1995).
36 Hung, M. C. On mammary gland growth factors: roles in normal development and in cancer. Cold Spring Harbor perspectives in biology 4, a013532 (2012).
37 Gerber, A. et al. Blood-borne circadian signal stimulates daily oscillations in actin dynamics and SRF activity. Cell 152, 492-503 (2013).
38 Cao, Y. et al. Glucocorticoid receptor translational isoforms underlie maturational stage-specific glucocorticoid sensitivities of dendritic cells in mice and humans. Blood 121, 1553-1562 (2013).
39 Sanchis, A., Bayo, P., Sevilla, L. M. & Perez, P. Glucocorticoid receptor antagonizes EGFR function to regulate eyelid development. The International journal of developmental biology 54, 1473-1480 (2010).
40 Odrowaz, Z. & Sharrocks, A. D. The ETS Transcription Factors ELK1 and GABPA Regulate Different Gene Networks to Control MCF10A Breast Epithelial Cell Migration. PloS one 7, doi:ARTN e49892 (2012).
41 Ayroldi, E. et al. Mechanisms of the anti-inflammatory effects of glucocorticoids: genomic and nongenomic interference with MAPK signalling pathways. FASEB journal: official publication of the Federation of American Societies for Experimental Biology 26, 4805-4820 (2012).
42 Alon, U. Network motifs: theory and experimental approaches. Nature reviews. Genetics 8, 450-461 (2007).
43 Amit, I. et al. A module of negative feedback regulators defines growth factor signalling. Nat Genet 39, 503-512 (2007).
44 Dorscheid, D. R. et al. Effects of corticosteroid-induced apoptosis on airway epithelial wound closure in vitro. American journal of physiology. Lung cellular and molecular physiology 291, L794-801 (2006).
45 Demaria, S. et al. Cancer and inflammation: promise for biologic therapy. J Immunother 33, 335-351 (2010).
46 Ulrich, C. M., Bigler, J. & Potter, J. D. Non-steroidal anti-inflammatory drugs for cancer prevention: promise, perils and pharmacogenetics. Nature reviews. Cancer 6, 130-140 (2006).
47 Ying, H. et al. Mig-6 controls EGFR trafficking and suppresses gliomagenesis. Proceedings of the National Academy of Sciences of the United States of America 107, 6912-6917 (2010).
48 Kramer, A. et al. Regulation of daily locomotor activity and sleep by hypothalamic EGF receptor signalling. Science 294, 2511-2515 (2001).
49 Sephton, S. E., Sapolsky, R. M., Kraemer, H. C. & Spiegel, D. Diurnal cortisol rhythm as a predictor of breast cancer survival. J Natl Cancer Inst 92, 994-1000 (2000).
50 Nakagawa, H. et al. Basis for dosing time-dependent change in the anti-tumor effect of imatinib in mice. Biochem Pharmacol 72, 1237-1245 (2006).
51 Zeisel, A., Yitzhaky, A., Bossel Ben-Moshe, N. & Domany, E. An accessible database for mouse and human whole transcriptome qPCR primers. Bioinformatics 29, 1355-1356 (2013).

1. A method of treating cancer in a subject in need thereof, the method comprising administering to the subject a receptor tyrosine kinase (RTK)-specific cancer therapy and a glucocorticoid or a glucocorticoid analog, such that an efficacy window of said RTK-specific cancer therapy and an efficacy window of said glucocorticoid or glucocorticoid analog substantially overlap.

2. A composition-of-matter comprising a therapeutically effective amount of an RTK-specific cancer therapy and a therapeutically effective amount of a glucocorticoid or glucocorticoid analog, the composition being such that an efficacy window of said RTK-specific cancer therapy and an efficacy window of said glucocorticoid or glucocorticoid analog substantially overlap.

3. An article of manufacture identified for the treatment of cancer comprising, in separate containers, a therapeutically effective amount of an RTK-specific cancer therapy and a therapeutically effective amount of a glucocorticoid or glucocorticoid analog.

4. The method of claim 1, wherein each of therapeutically effective amount of RTK-specific cancer therapy and therapeutically effective amount of said glucocorticoid or glucocorticoid analog is effective in treating cancer.

5. The method of claim 1, wherein said RTK-specific cancer therapy is conjugated to said glucocorticoid or glucocorticoid analog.

6. The method of claim 1, wherein said RTK-specific cancer therapy is administered paraenterally and/or wherein said glucocorticoid or analof is adminstered orally.

7. (canceled)

8. The method of claim 1, wherein said administering is under a circadian regimen.

9. The method of claim 8, wherein said regimen comprises administering said RTK-specific cancer therapy under glucocorticoid signalling activation.

10. (canceled)

11. The method of claim 1, wherein said glucocorticoid analog is selected from the group consisting of prednisone, prednisolone, fludrocortisone, and dexamethasone.

12. The method of claim 1, wherein said glucocorticoid analog comprises a non-steroidal glucocorticoid receptor agonist.

13. (canceled)

14. The method of claim 1, wherein said RTK-specific cancer therapy comprises a small molecule inhibitor or and antibody.

15. (canceled)

16. The method of claim 1, wherein said RTK is selected from the group consisting of c-met, VEGFR, INSR, PDGFR, EphR, FGFR and AXL.

17. The method of claim 1, wherein said RTK is an ErbB polypeptide.

18-20. (canceled)

21. The method of claim 1, wherein a maximal efficacy window of said RTK-specific cancer therapy and a maximal efficacy window of said glucocorticoid or glucocorticoid analog overlap for at least 10 hours.

22. The method of claim 1, wherein said RTK-specific cancer therapy and said glucocorticoid or glucocorticoid analog are administered substantially simultaneously.

23. The method of claim 1, being designed such that a plasma peak concentration of said RTK-specific cancer therapy and a plasma peak concentration of said glucocorticoid or glucocorticoid analog occur substantially simultaneously.

24. The method of claim 1, wherein said RTK-specific cancer therapy and said glucocorticoid or glucocorticoid analog are administered within 12 hours of each other.

25. The method of claim 1, wherein said RTK-specific cancer therapy and said glucocorticoid or glucocorticoid analog are administered within 1 hour of each other.

26. The method of claim 1, wherein said cancer is not a lymphoma, prostate cancer or breast cancer.

27-31. (canceled)

32. The method of claim 21, wherein said RTK-specific cancer therapy and said glucocorticoid or glucocorticoid analog are in a single formulation.

33-34. (canceled)


SEQUENCE LISTING (125 sequences)

SEQ ID NO: 1 - DNA, 20 bp, Artificial sequence
  Single strand DNA oligonucleotide
atactgcagcagggcagaac

SEQ ID NO: 2 - DNA, 25 bp, Artificial sequence
  Single strand DNA oligonucleotide
tttctgctttttactccctctgaat

SEQ ID NO: 3 - DNA, 20 bp, Artificial sequence
  Single strand DNA oligonucleotide
gagtcagatttccccctcgt

SEQ ID NO: 4 - DNA, 20 bp, Artificial sequence
  Single strand DNA oligonucleotide
agacgtgaccggattaacga

SEQ ID NO: 5 - DNA, 20 bp, Artificial sequence
  Single strand DNA oligonucleotide
cccaaaggtgggacttctct

SEQ ID NO: 6 - DNA, 21 bp, Artificial sequence
  Single strand DNA oligonucleotide
caagaaccactgctttttcca

SEQ ID NO: 7 - DNA, 19 bp, Artificial sequence
  Single strand DNA oligonucleotide
cgcactgcgaggagaagat

SEQ ID NO: 8 - DNA, 20 bp, Artificial sequence
  Single strand DNA oligonucleotide
acctcatcctgctccgtttc

SEQ ID NO: 9 - DNA, 20 bp, Artificial sequence
  Single strand DNA oligonucleotide
ttccaggcgttgtaccactt

SEQ ID NO: 10 - DNA, 20 bp, Artificial sequence
  Single strand DNA oligonucleotide
atcctgcatcccccatagtt

SEQ ID NO: 11 - DNA, 20 bp, Artificial sequence
  Single strand DNA oligonucleotide
gagcagggcaggagaagagt

SEQ ID NO: 12 - DNA, 20 bp, Artificial sequence
  Single strand DNA oligonucleotide
cttcaggaacagccaccagt

SEQ ID NO: 13 - DNA, 22 bp, Artificial sequence
  Single strand DNA oligonucleotide
acttcatagactccatcaagaa

SEQ ID NO: 14 - DNA, 20 bp, Artificial sequence
  Single strand DNA oligonucleotide
gaagggtgtttgtccactgc

SEQ ID NO: 15 - DNA, 20 bp, Artificial sequence
  Single strand DNA oligonucleotide
ctcgtccagcttgactcgat

SEQ ID NO: 16 - DNA, 19 bp, Artificial sequence
  Single strand DNA oligonucleotide
ccacagagcccttggacct

SEQ ID NO: 17 - DNA, 20 bp, Artificial sequence
  Single strand DNA oligonucleotide
cctgtgccaagcactttacc

SEQ ID NO: 18 - DNA, 20 bp, Artificial sequence
  Single strand DNA oligonucleotide
gaggaagggaaggatctcca

SEQ ID NO: 19 - DNA, 20 bp, Artificial sequence
  Single strand DNA oligonucleotide
tttgtcagcacctggaaaca

SEQ ID NO: 20 - DNA, 22 bp, Artificial sequence
  Single strand DNA oligonucleotide
catcacacaaatccttcatgct

SEQ ID NO: 21 - DNA, 20 bp, Artificial sequence
  Single strand DNA oligonucleotide
agacttggcctctccgaagt

SEQ ID NO: 22 - DNA, 20 bp, Artificial sequence
  Single strand DNA oligonucleotide
cgccaccaaactgagatgat

SEQ ID NO: 23 - DNA, 24 bp, Artificial sequence
  Single strand DNA oligonucleotide
gcctgttctaggacatcccaatta

SEQ ID NO: 24 - DNA, 20 bp, Artificial sequence
  Single strand DNA oligonucleotide
ttgtagtgggcagtgggatt

SEQ ID NO: 25 - DNA, 22 bp, Artificial sequence
  Single strand DNA oligonucleotide
tccatcttctacaggcagtcct

SEQ ID NO: 26 - DNA, 20 bp, Artificial sequence
  Single strand DNA oligonucleotide
cttccatgaaggctgcagaa

SEQ ID NO: 27 - DNA, 20 bp, Artificial sequence
  Single strand DNA oligonucleotide
agccacacgtggattgtctt

SEQ ID NO: 28 - DNA, 26 bp, Artificial sequence
  Single strand DNA oligonucleotide
tcctaatggaggtatttctgaattgt

SEQ ID NO: 29 - DNA, 20 bp, Artificial sequence
  Single strand DNA oligonucleotide
ctgggacatctccaaacctg

SEQ ID NO: 30 - DNA, 20 bp, Artificial sequence
  Single strand DNA oligonucleotide
cctcttcatgtggtcccaag

SEQ ID NO: 31 - DNA, 20 bp, Artificial sequence
  Single strand DNA oligonucleotide
gccactgctttgcagaaaat

SEQ ID NO: 32 - DNA, 20 bp, Artificial sequence
  Single strand DNA oligonucleotide
agctgctcggaacacttcag

SEQ ID NO: 33 - DNA, 24 bp, Artificial sequence
  Single strand DNA oligonucleotide
acctgatcagtttcccctatttct

SEQ ID NO: 34 - DNA, 20 bp, Artificial sequence
  Single strand DNA oligonucleotide
tgggcacacagtacagcaac

SEQ ID NO: 35 - DNA, 20 bp, Artificial sequence
  Single strand DNA oligonucleotide
tccctcctcatgtcatctcc

SEQ ID NO: 36 - DNA, 20 bp, Artificial sequence
  Single strand DNA oligonucleotide
cacggaagtgctgttgtcac

SEQ ID NO: 37 - DNA, 20 bp, Artificial sequence
  Single strand DNA oligonucleotide
tcggtgtgggaggttgtatt

SEQ ID NO: 38 - DNA, 20 bp, Artificial sequence
  Single strand DNA oligonucleotide
cttcaaggataagggcgaca

SEQ ID NO: 39 - DNA, 20 bp, Artificial sequence
  Single strand DNA oligonucleotide
ctcggttttggaccattctg

SEQ ID NO: 40 - DNA, 20 bp, Artificial sequence
  Single strand DNA oligonucleotide
tcttgccagttccctcattc

SEQ ID NO: 41 - DNA, 20 bp, Artificial sequence
  Single strand DNA oligonucleotide
gagctgggaagattcgaaca

SEQ ID NO: 42 - DNA, 20 bp, Artificial sequence
  Single strand DNA oligonucleotide
gttcctggaaaacggtaggc

SEQ ID NO: 43 - DNA, 20 bp, Artificial sequence
  Single strand DNA oligonucleotide
cgagagatacgcaggtgcag

SEQ ID NO: 44 - DNA, 20 bp, Artificial sequence
  Single strand DNA oligonucleotide
cctgtgccaagtgtgtgaga

SEQ ID NO: 45 - DNA, 20 bp, Artificial sequence
  Single strand DNA oligonucleotide
cggctggagtacagaggcta

SEQ ID NO: 46 - DNA, 22 bp, Artificial sequence
  Single strand DNA oligonucleotide
gacttcgtgattattcccatcg

SEQ ID NO: 47 - DNA, 20 bp, Artificial sequence
  Single strand DNA oligonucleotide
gctgtggtgctgtcatctgt

SEQ ID NO: 48 - DNA, 20 bp, Artificial sequence
  Single strand DNA oligonucleotide
ctttggaaggacctgctctg

SEQ ID NO: 49 - DNA, 21 bp, Artificial sequence
  Single strand DNA oligonucleotide
tcatgcccaacttcactttct

SEQ ID NO: 50 - DNA, 20 bp, Artificial sequence
  Single strand DNA oligonucleotide
cggaaggaaccatctcactg

SEQ ID NO: 51 - DNA, 21 bp, Artificial sequence
  Single strand DNA oligonucleotide
aaaggaagtagctggcagagc

SEQ ID NO: 52 - DNA, 20 bp, Artificial sequence
  Single strand DNA oligonucleotide
agcactccttggcaaaactg

SEQ ID NO: 53 - DNA, 20 bp, Artificial sequence
  Single strand DNA oligonucleotide
aaggcggacattctggaaat

SEQ ID NO: 54 - DNA, 21 bp, Artificial sequence
  Single strand DNA oligonucleotide
tgacccgtctgtctctttctg

SEQ ID NO: 55 - DNA, 20 bp, Artificial sequence
  Single strand DNA oligonucleotide
tacttccccagcacacttgg

SEQ ID NO: 56 - DNA, 20 bp, Artificial sequence
  Single strand DNA oligonucleotide
ggactacgctctggacctca

SEQ ID NO: 57 - DNA, 20 bp, Artificial sequence
  Single strand DNA oligonucleotide
agtgcggggagtcacagtta

SEQ ID NO: 58 - DNA, 20 bp, Artificial sequence
  Single strand DNA oligonucleotide
cgacctgacctgtctcctgt

SEQ ID NO: 59 - DNA, 21 bp, Artificial sequence
  Single strand DNA oligonucleotide
gcagaaagagaagccttttgg

SEQ ID NO: 60 - DNA, 20 bp, Artificial sequence
  Single strand DNA oligonucleotide
gccagagctgtgcagatgag

SEQ ID NO: 61 - DNA, 20 bp, Artificial sequence
  Single strand DNA oligonucleotide
catcatcccatagcccagag

SEQ ID NO: 62 - DNA, 20 bp, Artificial sequence
  Single strand DNA oligonucleotide
tcaggggtggttattgcatc

SEQ ID NO: 63 - DNA, 27 bp, Artificial sequence
  Single strand DNA oligonucleotide
tcatagctctactgatttcttctctgg

SEQ ID NO: 64 - DNA, 22 bp, Artificial sequence
  Single strand DNA oligonucleotide
cgaacatcaatttcatttgcag

SEQ ID NO: 65 - DNA, 20 bp, Artificial sequence
  Single strand DNA oligonucleotide
attgcttccacccttcttcc

SEQ ID NO: 66 - DNA, 20 bp, Artificial sequence
  Single strand DNA oligonucleotide
aggacagggacgaacatcaa

SEQ ID NO: 67 - DNA, 20 bp, Artificial sequence
  Single strand DNA oligonucleotide
cgctgtgacattcgctacac

SEQ ID NO: 68 - DNA, 27 bp, Artificial sequence
  Single strand DNA oligonucleotide
aaaggttgactttaaatttgtctgttg

SEQ ID NO: 69 - DNA, 20 bp, Artificial sequence
  Single strand DNA oligonucleotide
ccattgggagttttgctttg

SEQ ID NO: 70 - DNA, 24 bp, Artificial sequence
  Single strand DNA oligonucleotide
tctgaccaatttttctctttttgc

SEQ ID NO: 71 - DNA, 20 bp, Artificial sequence
  Single strand DNA oligonucleotide
ggacagggttggaggaagaa

SEQ ID NO: 72 - DNA, 20 bp, Artificial sequence
  Single strand DNA oligonucleotide
tgcccagctccttagacaag

SEQ ID NO: 73 - DNA, 20 bp, Artificial sequence
  Single strand DNA oligonucleotide
ccgaaagaagcaagctgaag

SEQ ID NO: 74 - DNA, 21 bp, Artificial sequence
  Single strand DNA oligonucleotide
tggctaattctgtgccttttg

SEQ ID NO: 75 - DNA, 21 bp, Artificial sequence
  Single strand DNA oligonucleotide
tgctgtgtcttggttttcctc

SEQ ID NO: 76 - DNA, 21 bp, Artificial sequence
  Single strand DNA oligonucleotide
tggttcaagaatgggaatgaa

SEQ ID NO: 77 - DNA, 20 bp, Artificial sequence
  Single strand DNA oligonucleotide
tgacaccactttggtcctga

SEQ ID NO: 78 - DNA, 21 bp, Artificial sequence
  Single strand DNA oligonucleotide
ctctccagaatcagccagtga

SEQ ID NO: 79 - DNA, 19 bp, Artificial sequence
  Single strand DNA oligonucleotide
tgttgcaccaggttcttcg

SEQ ID NO: 80 - DNA, 20 bp, Artificial sequence
  Single strand DNA oligonucleotide
ggtgggattttgttgtttgc

SEQ ID NO: 81 - DNA, 20 bp, Artificial sequence
  Single strand DNA oligonucleotide
ggcaaactgctctgcaagat

SEQ ID NO: 82 - DNA, 21 bp, Artificial sequence
  Single strand DNA oligonucleotide
ctccgtaggcttcaagaccat

SEQ ID NO: 83 - DNA, 20 bp, Artificial sequence
  Single strand DNA oligonucleotide
cattccttccccctaaccag

SEQ ID NO: 84 - DNA, 20 bp, Artificial sequence
  Single strand DNA oligonucleotide
tctccaggacagcttccatc

SEQ ID NO: 85 - DNA, 20 bp, Artificial sequence
  Single strand DNA oligonucleotide
aacactggcaatgtcaagca

SEQ ID NO: 86 - DNA, 20 bp, Artificial sequence
  Single strand DNA oligonucleotide
tcaacacagctagggcatga

SEQ ID NO: 87 - DNA, 20 bp, Artificial sequence
  Single strand DNA oligonucleotide
ttgtcctggcaacgttttct

SEQ ID NO: 88 - DNA, 24 bp, Artificial sequence
  Single strand DNA oligonucleotide
tttgatggctactcagaagattca

SEQ ID NO: 89 - DNA, 21 bp, Artificial sequence
  Single strand DNA oligonucleotide
tgggtcaagactcaaaccaaa

SEQ ID NO: 90 - DNA, 22 bp, Artificial sequence
  Single strand DNA oligonucleotide
tggtatccccatctacagaacc

SEQ ID NO: 91 - DNA, 20 bp, Artificial sequence
  Single strand DNA oligonucleotide
caatgaacttgtggcctgtg

SEQ ID NO: 92 - DNA, 20 bp, Artificial sequence
  Single strand DNA oligonucleotide
tcaaccagctgggcatagtt

SEQ ID NO: 93 - DNA, 21 bp, Artificial sequence
  Single strand DNA oligonucleotide
agacccaaggatgagttccag

SEQ ID NO: 94 - DNA, 18 bp, Artificial sequence
  Single strand DNA oligonucleotide
ggcgtctgcgagtacagc

SEQ ID NO: 95 - DNA, 20 bp, Artificial sequence
  Single strand DNA oligonucleotide
ggattaggcatcctgctcaa

SEQ ID NO: 96 - DNA, 20 bp, Artificial sequence
  Single strand DNA oligonucleotide
ctgagcatcaggctgcaaac

SEQ ID NO: 97 - DNA, 20 bp, Artificial sequence
  Single strand DNA oligonucleotide
aggaggagggcagaatcatc

SEQ ID NO: 98 - DNA, 20 bp, Artificial sequence
  Single strand DNA oligonucleotide
gcattacagagctgggtgga

SEQ ID NO: 99 - DNA, 20 bp, Artificial sequence
  Single strand DNA oligonucleotide
agctgcgctgatagacatcc

SEQ ID NO: 100 - DNA, 20 bp, Artificial sequence
  Single strand DNA oligonucleotide
accctggaaagccagaagaa

SEQ ID NO: 101 - DNA, 21 bp, Artificial sequence
  Single strand DNA oligonucleotide
tgctgggtcttacatgcagat

SEQ ID NO: 102 - DNA, 20 bp, Artificial sequence
  Single strand DNA oligonucleotide
ctgaacgccccacatgtact

SEQ ID NO: 103 - DNA, 21 bp, Artificial sequence
  Single strand DNA oligonucleotide
cttcacacgccaagaaacagt

SEQ ID NO: 104 - DNA, 20 bp, Artificial sequence
  Single strand DNA oligonucleotide
gctggcccatagtgatcttt

SEQ ID NO: 105 - DNA, 21 bp, Artificial sequence
  Single strand DNA oligonucleotide
ccctggagagctagggtaaca

SEQ ID NO: 106 - DNA, 20 bp, Artificial sequence
  Single strand DNA oligonucleotide
gtttttggtgcaggaggaca

SEQ ID NO: 107 - DNA, 20 bp, Artificial sequence
  Single strand DNA oligonucleotide
caccaacgtacccagaatgg

SEQ ID NO: 108 - protein, 705 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 109 - protein, 1255 aa, Homo sapiens [sequence omitted]

SEQ ID NO: 110 - protein, 183 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 111 - protein, 1308 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 112 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 113 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 114 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 115 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 116 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 117 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 118 - protein, 987 aa, Homo sapiens
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 119 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 120 - protein, 366 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQGRRC

SEQ ID NO: 121 - protein, 694 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 122 - protein, 913 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 123 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 124 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 125 - protein, 885 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA